(19) United States
(12) Patent Application Publication
(10) Pub. No.: US 2025/0037823 A1
(43) Pub. Date: Jan. 30, 2025

(54) METHODS AND SYSTEMS FOR HIGHLIGHTING CLINICAL INFORMATION IN DIAGNOSTIC REPORTS

(71) Applicant: Foundation Medicine, Inc., Boston, MA (US)

(72) Inventors: Rachel ERBACH, Arlington, MA (US); Lauren THORPE, Brighton, MA (US); Erica GORNSTEIN, Cambridge, MA (US)

(73) Assignee: Foundation Medicine, Inc., Boston, MA (US)

(21) Appl. No.: 18/717,942

(22) PCT Filed: Dec. 2, 2022
(86) PCT No.: PCT/US2022/080862

§ 371 Date: Jun. 7, 2024

Related U.S. Application Data
(60) Provisional application No. 63/287,475, filed on Dec. 8, 2021.

(51) Int. Cl. G16H 15/00; G16H 10/60; G16H 50/20

(52) U.S. Cl. CPC G16H 15/00; G16H 10/60; G16H 50/20

(57) ABSTRACT

Methods for generating a report of genomic and medical information associated with a patient are described. The methods may comprise, for example, receiving genomic testing data associated with the patient. The methods may comprise, for example, based on the genomic testing data, retrieving medical information including one or more potential clinical treatments for the patient. The methods may comprise, for example, determining that the medical information has at least some clinical significance to the patient. The methods may comprise, for example, based on at least a portion of the medical information having at least some clinical significance to the patient, generating patient-specific medical data. The methods may comprise, for example, determining at least one specific position to dispose the medical information on the report based on the patient-specific medical data and generating the report based on the determined at least one specific position.

### CROSS-REFERENCE TO RELATED APPLICATIONS

This application claims the benefit of U.S. Provisional Application No. 63/287,475, filed Dec. 8, 2021, the entire contents of which are incorporated herein by reference in their entirety.

### FIELD OF THE INVENTION

The present disclosure relates generally to methods and systems for analyzing genomic profiling data, and more specifically to methods and systems for highlighting clinically relevant information in diagnostics reports using genomic profiling data.

### BACKGROUND

Genomic sequencing clinical reports may generally provide genomic and medical information that may be specific to a particular patient and the particular type of disease (e.g., cancer) from which that particular patient is suffering. The genomic and medical information generally covers a breadth of topics. However, because the genomic and medical information, and associated clinical information, may often be presented within the clinical report in a counterintuitive and/or deemphasized manner, clinicians and patients alike may struggle to distinguish genomic and medical information (e.g., clinical information) of relevance (e.g., of high clinical importance) from that of less relevance (e.g., less clinical importance) to the patient. This may lead to clinicians and patients overlooking clinical information within the clinical reports, and thus potentially decreasing the overall efficacy of care provided to cancer patients. It may be useful to provide techniques to improve genomic sequencing clinical reports.

### BRIEF SUMMARY OF THE INVENTION

Disclosed herein are methods and systems for generating a report of genomic and medical information associated with a patient, in which the report automatically highlights and emphasizes clinical genomic and medical information to allow clinicians and patients to more readily ascertain genomic and medical information having the highest impact to patient care, and, by extension, improves the overall efficacy of care provided to patients. In certain embodiments, one or more processors may receive, at the one or more processors, genomic testing data associated with a patient. In certain embodiments, the one or more processors may, based on the genomic testing data, retrieve, at the one or more processors, medical information including one or more potential clinical treatments for the patient. In certain embodiments, one or more processors may determine, by the one or more processors, that the medical information has at least some clinical significance to the patient. In certain embodiments, one or more processors may, based on at least a portion of the medical information having at least some clinical significance to the patient, generate, by the one or more processors, patient-specific medical data. In certain embodiments, the one or more processors may determine, by the one or more processors, at least one specific position to dispose the medical information on the report based on the patient-specific medical data. In certain embodiments, one or more processors may generate, by the one or more processors, the report based on the determined specific position.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a genomic sequencing clinical report.

FIG. 2A provides a non-limiting example of a flow diagram of a process for generating a report of genomic and medical information associated with a patient.

FIG. 2B illustrates a non-limiting example of a computing architecture for generating a report of genomic and medical information associated with a patient.

FIG. 2C provides a non-limiting example of a flow diagram of a process for generating and storing a number of annotated data objects to a knowledge base.

FIG. 2D illustrates an example embodiment of a knowledge base that may include a predetermined set of categories and annotated data objects.

FIG. 2E provides a non-limiting example of a flow diagram of a process for populating a report highlighting pane of a report associated with a patient with one or more annotated data objects.

FIG. 2F illustrates an example of a report highlighting pane, including each category of an example predetermined set of treatment or therapeutic categories that may be included in the report highlighting pane.

FIG. 3A provides a non-limiting example of a genomic sequencing clinical report without including a report highlighting pane.

FIGS. 3B and 3C provide non-limiting running examples of genomic sequencing clinical reports including a report highlighting pane.

FIG. 3D illustrates a non-limiting example of one or more pages of a genomic sequencing clinical report with a report highlighting pane.

FIG. 3E illustrates a non-limiting example of an appendix page of a genomic sequencing clinical report with a report highlighting pane.

FIG. 3F illustrates a non-limiting example of a genomic sequencing clinical report with a report highlighting pane.

FIG. 4 depicts an exemplary computing device or system in accordance with one embodiment of the present disclosure.

FIG. 5 depicts an exemplary computer system or computer network, in accordance with some instances of the systems described herein.

### DETAILED DESCRIPTION

Methods and systems for generating a report of genomic and medical information associated with a patient, in which the report automatically highlights and emphasizes the genomic and medical information to allow clinicians and patients to more readily ascertain genomic and medical information having the highest impact to patient care, and, by extension, improves the overall efficacy of care provided to patients. In certain embodiments, one or more processors may receive, at the one or more processors, genomic testing data associated with a patient. In certain embodiments, the one or more processors may, based on the genomic testing data, retrieve, at the one or more processors, medical information including one or more potential clinical treatments for the patient. In certain embodiments, one or more processors may determine, by the one or more processors, that the medical information has at least some clinical significance to the patient. In certain embodiments, one or more processors may, based on at least a portion of the medical information having at least some clinical significance to the patient, generate, by the one or more processors, patient-specific medical data. In certain embodiments, the one or more processors may determine, by the one or more processors, at least one specific position to dispose the medical information on the report based on the patient-specific medical data. In certain embodiments, one or more processors may generate, by the one or more processors, the report based on the determined specific position.

In certain embodiments, the one or more processors may obtain genomic testing data associated with the patient. Obtaining the genomic testing data comprises obtaining biomarker testing data, tumor testing data, molecular testing data, next-generation sequencing (NGS) data, or genomic profiling data, previous treatments, clinical history, clinical status, and so forth. In certain embodiments, the one or more processors may apply the number of rulesets against the set of genomic and medical data to obtain a set of one or more objects by applying each of the number of rulesets against the set of genomic and medical data to determine a match to the set of one or more objects and retrieving the set of one or more objects based on the determined match. In certain embodiments, the one or more processors may apply the number of rulesets against the set of genomic and medical data which may further include applying a number of rulesets corresponding to a predetermined set of genomic or therapeutic categories. In certain embodiments, the set of one or more objects are ranked according to relative clinical significances, in which the relative clinical significances of the predetermined set are predefined. In certain embodiments, the predetermined set of genomic or therapeutic categories may include a genomic findings with diagnostic implications category, a targeted therapies with highest level of evidence category, a targeted therapies with expected resistance category, a genomic findings with non-targeted therapy implications category, an evidence-matched clinical trial options category, a genomic findings with prognostic implications category, a genomic findings with germline implications category, a genomic findings with clonal hematopoiesis (CH) implications category, a genomic findings with companion diagnostic (CDx) category, a targeted therapies with pan-tumor indications category, or an investigational targeted therapies category. In certain embodiments, the one or more processors may determine the clinical significance of at least a portion of the set of genomic and medical data by obtaining a set of scientific and medical literature and inputting the set of scientific and medical literature to a trained machine-learning model to obtain a prediction of the set of one or more objects based on the set of scientific and medical literature.

In certain embodiments, the one or more processors may further apply the number of rulesets against the genomic testing data by comparing the set of genomic and medical data against a respective ruleset corresponding to genomic findings with diagnostic implications and determining whether the set of genomic and medical data satisfies a first predetermined criteria based on the genomic findings with diagnostic implications. In one embodiment, the first predetermined criteria includes a match of a gene variant and a patient tumor type to a first object in the knowledge base corresponding to the genomic findings with diagnostic implications. In certain embodiments, based on a determination that the set of genomic and medical data satisfies the first predetermined criteria, the one or more processors may display the genomic findings with diagnostic implications in a first predefined space of the predefined area. In certain embodiments, based on a determination that the set of genomic and medical data fails to satisfy the first predetermined criteria, forgoing displaying the genomic findings with diagnostic implications in the first predefined space. In certain embodiments, the one or more processors may generate the first object in the knowledge base, in which the first object includes an annotation indicative of a gene variant and a tumor type. In one embodiment, the first predetermined criteria includes a match of the gene variant and patient tumor type to the annotation of the first object.

In certain embodiments, the one or more processors may further apply the number of rulesets against the set of genomic and medical data by comparing against a respective ruleset corresponding to targeted therapies, and determining whether the set of genomic and medical data satisfies a second predetermined criteria based on the targeted therapies. In one embodiment, the second predetermined criteria includes a match of the targeted therapies to a second object in the knowledge base corresponding to one or more genomic findings or biomarker findings. In certain embodiments, based on a determination that the set of genomic and medical data satisfies the second predetermined criteria, the one or more processors may display targeted therapies with a highest level of evidence in a second predefined space of the predefined area. In certain embodiments, based on a determination that the set of genomic and medical data fails to satisfy the predetermined criteria, the one or more processors may forgo displaying the targeted therapies with the highest level of evidence in the second predefined space. In certain embodiments, the one or more processors may generate the second object in the knowledge base, in which the second object includes an annotation indicative of the one or more genomic findings or biomarker findings. In one embodiment, the second predetermined criteria includes a match of the targeted therapies to the annotation of the second object. In certain embodiments, based on a determination that the set of genomic and medical data fails to satisfy the first predetermined criteria, the one or more processors may display the targeted therapies with highest level of evidence in the first predefined space of the predefined area.

In certain embodiments, the one or more processors may further apply the number of rulesets against the set of genomic and medical data by comparing the set of genomic and medical data against a respective ruleset corresponding to targeted therapies, and determining whether the set of genomic and medical data satisfies a third predetermined criteria based on the targeted therapies. In one embodiment, the third predetermined criteria includes a match of the targeted therapies to a third object in the knowledge base corresponding to therapies resistant to the one or more genomic findings or biomarker findings. In certain embodiments, based on a determination that the set of genomic and medical data satisfies the third predetermined criteria, the one or more processors may display the targeted therapies with expected resistance in a third predefined space of the predefined area. In certain embodiments, based on a determination that the set of genomic and medical data fails to satisfy the third predetermined criteria, the one or more processors may forgo displaying the targeted therapies with expected resistance in the third predefined space. In certain embodiments, the one or more processors may generate the third object in the knowledge base, in which the third object includes an annotation indicative of the therapies resistant to the one or more genomic findings or biomarker findings. In one embodiment, the third predetermined criteria includes a match of the targeted therapies to the annotation of the third object. In certain embodiments, based on a determination that the set of genomic and medical data fails to satisfy the first predetermined criteria or the second predetermined criteria, the one or more processors may display the targeted therapies with expected resistance in the first predefined space or the second predefined space.

In certain embodiments, the one or more processors may further apply the number of rulesets against the set of genomic and medical data by comparing the set of genomic and medical data against a respective ruleset corresponding to genomic findings with non-targeted therapy implications, and determining whether the set of genomic and medical data satisfies a fourth predetermined criteria based on the genomic findings with non-targeted therapy implications. In one embodiment, the fourth predetermined criteria includes a match of a gene variant and a patient tumor type to a fourth object in the knowledge base corresponding to the genomic findings with non-targeted therapy implications. In certain embodiments, based on a determination that the set of genomic and medical data satisfies the fourth predetermined criteria, the one or more processors may display the genomic findings with non-targeted therapy implications in a fourth predefined space of the predefined area. In certain embodiments, based on a determination that the set of genomic and medical data fails to satisfy the fourth predetermined criteria, the one or more processors may forgo displaying the genomic findings with non-targeted therapy implications in the fourth predefined space. In certain embodiments, the one or more processors may generate the fourth object in the knowledge base, in which the fourth object includes an annotation indicative of a gene variant and a tumor type. In one embodiment, the fourth predetermined criteria includes a match of the gene variant and patient tumor type to the annotation of the fourth object.

In certain embodiments, the one or more processors may further apply the number of rulesets against the set of genomic and medical data by comparing the set of genomic and medical data against a respective ruleset corresponding to clinical trials, and determining whether the set of genomic and medical data satisfies a fifth predetermined criteria based on the clinical trials. In one embodiment, the fifth predetermined criteria includes a sensitivity to a targeted therapy type. In certain embodiments, based on a determination that the set of genomic and medical data satisfies the fifth predetermined criteria, the one or more processors may display clinical trial options in a fifth predefined space of the predefined area. In certain embodiments, based on a determination that the set of genomic and medical data fails to satisfy the fifth predetermined criteria, the one or more processors may forgo displaying the clinical trial options in the fifth predefined space. In certain embodiments, the one or more processors may generate the fifth object in the knowledge base, in which the fifth object includes an annotation indicative of a gene variant, a tumor type, patient age, biomarker findings, genomic signatures, and/or a clinical trial patient recruitment criteria. In one embodiment, the fifth predetermined criteria includes a match of the gene variant and patient tumor type to the annotation of the fifth object.

In certain embodiments, the one or more processors may further apply the number of rulesets against the set of genomic and medical data by comparing the set of genomic and medical data against a respective ruleset corresponding to genomic findings with prognostic implications, and determining whether the set of genomic and medical data satisfies a sixth predetermined criteria based on the genomic findings with prognostic implications. In one embodiment, the sixth predetermined criteria includes a match of a gene variant and patient tumor type to a sixth object in the knowledge base corresponding to the genomic findings with prognostic implications. In certain embodiments, based on a determination that the set of genomic and medical data satisfies the sixth predetermined criteria, the one or more processors may display the genomic findings with prognostic implications in a sixth predefined space of the predefined area. In certain embodiments, based on a determination that the set of genomic and medical data fails to satisfy the sixth predetermined criteria, the one or more processors may forgo displaying the genomic findings with prognostic implications in the sixth predefined space. In certain embodiments, the one or more processors may generate the sixth object in the knowledge base, in which the sixth object includes an annotation indicative of a gene variant and a tumor type. In one embodiment, the sixth predetermined criteria includes a match of the gene variant and patient tumor type to the annotation of the sixth object.

In certain embodiments, the one or more processors may further apply the number of rulesets against the set of genomic and medical data by comparing the set of genomic and medical data against a respective ruleset corresponding to genomic findings with germline implications, and determining whether the set of genomic and medical data satisfies a seventh predetermined criteria based on the genomic findings with potential germline implications. In certain embodiments, the seventh predetermined criteria includes: 1) a match of a potential germline mutation associated with one or more hereditary cancer predisposition syndromes to a seventh object in the knowledge base corresponding to the genomic findings with germline implications, and 2) a determination that the mutation is above a predetermined variant allele frequency (VAF) threshold. In certain embodiments, based on a determination that the set of genomic and medical data satisfies the seventh predetermined criteria, the one or more processors may display the genomic findings with germline implications in a seventh predefined space of the predefined area. In certain embodiments, based on a determination that the set of genomic and medical data fails to satisfy the seventh predetermined criteria, the one or more processors may forgo displaying the genomic findings with germline implications in the seventh predefined space. In certain embodiments, the one or more processors may generate the seventh object in the knowledge base, in which the seventh object includes an annotation indicative of a gene variant, a tumor type, biomarker findings, and/or genomic signatures. In one embodiment, the seventh predetermined criteria comprises a match of the potential germline mutation to the annotation of the seventh object.

In certain embodiments, the one or more processors may further apply the number of rulesets against the genomic testing data by comparing the set of genomic and medical data against a respective ruleset corresponding to genomic findings with clonal hematopoiesis (CH) implications, and determining whether the set of genomic and medical data satisfies an eighth predetermined criteria based on the genomic findings with CH implications. In certain embodiments, the eighth predetermined criteria includes: 1) a match of a CH to an eighth object in the knowledge base corresponding to the genomic findings with CH implications, and 2) a determination that the CH is associated with one or more particular analytes. In certain embodiments, based on a determination that the set of genomic and medical data satisfies the eighth predetermined criteria, the one or more processors may display the genomic findings with CH implications in an eighth predefined space of the predefined area. In certain embodiments, based on a determination that the set of genomic and medical data fails to satisfy the eighth predetermined criteria, the one or more processors may forgo displaying the genomic findings with CH implications in the eighth predefined space. In certain embodiments, the one or more processors may generate the eighth object in the knowledge base, in which the eighth object includes an annotation indicative of a gene variant and a tumor type. In one embodiment, the eighth predetermined criteria includes a match of the CH to the annotation of the eighth object.

In certain embodiments, the one or more processors may cause an electronic device to display the report by causing the electronic device to display the report including the predefined area for the one or more natural-language phrases on a first page of the report. In certain embodiments, the one or more natural-language phrases may include an embedded hyperlink associated with the one or more natural-language phrases. In certain embodiments, the embedded hyperlink may include a link to one or more other pages of the report, in which the one or more other pages include a set of detailed information corresponding to the one or more natural-language phrases. In certain embodiments, the embedded hyperlink may include a page number of the one or more other pages. In certain embodiments, the one or more processors may generate the report by generating a companion diagnostic (CDx) report associated with the patient. In certain embodiments, the one or more processors may cause an electronic device to display the report by causing a human machine interface (HMI) associated with a clinician to display the report. In certain embodiments, the one or more processors may cause an electronic device to display the report by causing an electronic device associated with the patient to display the report. In certain embodiments, the one or more processors may further cause the electronic device to display the report including the one or more natural-language phrases in a second predefined area. In certain embodiments, the second predefined area includes an appendix on one or more rearward pages of the report. In certain embodiments, the appendix may include the one or more natural-language phrases displayed along with additional contextual information, such as that information determined to be relevant to the patient.

The disclosed methods and systems thus generates a report of genomic and medical information associated with a patient, in which the report automatically highlights and emphasizes the most clinically significant genomic and medical information. This may allow clinicians and patients to more readily ascertain genomic and medical information having the highest impact to patient care, and, by extension, improve the overall efficacy of care provided to patients.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

The terms “clinically relevant” and “clinically significant” are used interchangeably and refer to any information relevant to diagnosis and/or treatment of a disease (e.g., cancer) of a patient, including but not limited to information that may be of a highest priority or relevance to the diagnosis and/or treatment of the disease based on a disease state of the patient.

The term “medical information” refers to one or more therapeutic, diagnostic, prognostic, potential germline, potential clonal hematopoiesis, or other related information based, at least in part, on a patient's medical information.

The term “patient-specific medical data” refers to one or more therapeutic, diagnostic, prognostic, potential germline, potential clonal hematopoiesis, or other related data having a clinical relevance to a patient based, at least in part, on a patient's genomic information.

The section headings used herein are for organizational purposes only and are not to be construed as limiting the subject matter described.

Methods for Generating a Report of Genomic and Medical Information Associated with a Patient

The disclosed methods for generating a report of genomic and medical information associated with a patient, in which the report automatically highlights and emphasizes the genomic and medical information to allow clinicians and patients to more readily ascertain genomic and medical information having the highest impact to patient care, and, by extension, improves the overall efficacy of care provided to patients. In certain embodiments, one or more processors may receive, at the one or more processors, genomic testing data associated with a patient. In certain embodiments, the one or more processors may, based on the genomic testing data, retrieve, at the one or more processors, medical information including one or more potential clinical treatments for the patient. In certain embodiments, one or more processors may determine, by the one or more processors, that the medical information has at least some clinical significance to the patient. In certain embodiments, one or more processors may, based on at least a portion of the medical information having at least some clinical significance to the patient, generate, by the one or more processors, patient-specific medical data. In certain embodiments, the one or more processors may determine, by the one or more processors, at least one specific position to dispose the medical information on the report based on the patient-specific medical data. In certain embodiments, one or more processors may generate, by the one or more processors, the report based on the determined specific position.

In certain embodiments, the one or more processors may obtain genomic testing data associated with the patient by obtaining the genomic testing data comprises obtaining biomarker testing data, tumor testing data, molecular testing data, next-generation sequencing (NGS) data, or genomic profiling data. In certain embodiments, the one or more processors may apply the number of rulesets against the set of genomic and medical data to obtain a set of one or more objects by applying each of the number of rulesets against the set of genomic and medical data to determine a match to the set of one or more objects and retrieving the set of one or more objects based on the determined match. In certain embodiments, the one or more processors may apply the number of rulesets against the set of genomic and medical data may further include applying a number of rulesets corresponding to a predetermined set of genomic or therapeutic categories. In certain embodiments, the set of one or more objects are ranked according to relative clinical significances, in which the relative clinical significances of the predetermined set are predefined. In certain embodiments, the predetermined set of genomic or therapeutic categories may include a genomic findings with diagnostic implications category, a targeted therapies with highest level of evidence category, a targeted therapies with expected resistance category, a genomic findings with non-targeted therapy implications category, an evidence-matched clinical trial options category, a genomic findings with prognostic implications category, a genomic findings with germline implications category, a genomic findings with clonal hematopoiesis (CH) implications category, a genomic findings with companion diagnostic (CDx) category, a targeted therapies with pan-tumor indications category, or an investigational targeted therapies category. In certain embodiments, the one or more processors may determine the clinical significance of at least a portion of the set of genomic and medical data by obtaining a set of scientific and medical literature and inputting the set of scientific and medical literature to a trained machine-learning model to obtain a prediction of the set of one or more objects based on the set of scientific and medical literature.

In certain embodiments, the one or more processors may further apply the number of rulesets against the genomic testing data by comparing the set of genomic and medical data against a respective ruleset corresponding to genomic findings with diagnostic implications, and determining whether the set of genomic and medical data satisfies a first predetermined criteria based on the genomic findings with diagnostic implications. In one embodiment, the first predetermined criteria includes a match of a gene variant and a patient tumor type to a first object in the knowledge base corresponding to the genomic findings with diagnostic implications. In certain embodiments, based on a determination that the set of genomic and medical data satisfies the first predetermined criteria, the one or more processors may display the genomic findings with diagnostic implications in a first predefined space of the predefined area. In certain embodiments, based on a determination that the set of genomic and medical data fails to satisfy the first predetermined criteria, forgoing displaying the genomic findings with diagnostic implications in the first predefined space. In certain embodiments, the one or more processors may generate the first object in the knowledge base, in which the first object including an annotation indicative of a gene variant and a tumor type. In one embodiment, the first predetermined criteria includes a match of the gene variant and patient tumor type to the annotation of the first object.

In certain embodiments, the one or more processors may further apply the number of rulesets against the set of genomic and medical data by comparing against a respective ruleset corresponding to targeted therapies, and determining whether the set of genomic and medical data satisfies a second predetermined criteria based on the targeted therapies. In one embodiment, the second predetermined criteria includes a match of the targeted therapies to a second object in the knowledge base corresponding to one or more genomic findings or biomarker findings. In certain embodiments, based on a determination that the set of genomic and medical data satisfies the second predetermined criteria, the one or more processors may display targeted therapies with a highest level of evidence in a second predefined space of the predefined area. In certain embodiments, based on a determination that the set of genomic and medical data fails to satisfy the predetermined criteria, the one or more processors may forgo displaying the targeted therapies with the highest level of evidence in the second predefined space. In certain embodiments, the one or more processors may generate the second object in the knowledge base, in which the second object includes an annotation indicative of the one or more genomic findings or biomarker findings. In one embodiment, the second predetermined criteria includes a match of the targeted therapies to the annotation of the second object. In certain embodiments, based on a determination that the set of genomic and medical data fails to satisfy the first predetermined criteria, the one or more processors may display the targeted therapies with highest level of evidence in the first predefined space of the predefined area.

In certain embodiments, the one or more processors may further apply the number of rulesets against the set of genomic and medical data by comparing the set of genomic and medical data against a respective ruleset corresponding to targeted therapies, and determining whether the set of genomic and medical data satisfies a third predetermined criteria based on the targeted therapies. In one embodiment, the third predetermined criteria includes a match of the targeted therapies to a third object in the knowledge base corresponding to therapies resistant to the one or more genomic findings or biomarker findings. In certain embodiments, based on a determination that the set of genomic and medical data satisfies the third predetermined criteria, the one or more processors may display the targeted therapies with expected resistance in a third predefined space of the predefined area. In certain embodiments, based on a determination that the set of genomic and medical data fails to satisfy the third predetermined criteria, the one or more processors may forgo displaying the targeted therapies with expected resistance in the third predefined space. In certain embodiments, the one or more processors may generate the third object in the knowledge base, in which the third object includes an annotation indicative of the therapies resistant to the one or more genomic findings or biomarker findings. In one embodiment, the third predetermined criteria includes a match of the targeted therapies to the annotation of the third object. In certain embodiments, based on a determination that the set of genomic and medical data fails to satisfy the first predetermined criteria or the second predetermined criteria, the one or more processors may display the targeted therapies with expected resistance in the first predefined space or the second predefined space.

In certain embodiments, the one or more processors may further apply the number of rulesets against the set of genomic and medical data by comparing the set of genomic and medical data against a respective ruleset corresponding to genomic findings with non-targeted therapy, and determining whether the set of genomic and medical data satisfies a fourth predetermined criteria based on the genomic findings with non-targeted therapy. In one embodiment, the fourth predetermined criteria includes a match of a gene variant and a patient tumor type to a fourth object in the knowledge base corresponding to the genomic findings with non-targeted therapy. In certain embodiments, based on a determination that the set of genomic and medical data satisfies the fourth predetermined criteria, the one or more processors may display the genomic findings with non-targeted therapy in a fourth predefined space of the predefined area. In certain embodiments, based on a determination that the set of genomic and medical data fails to satisfy the fourth predetermined criteria, the one or more processors may forgo displaying the genomic findings with non-targeted therapy in the fourth predefined space. In certain embodiments, the one or more processors may generate the fourth object in the knowledge base, in which the fourth object includes an annotation indicative of a gene variant and a tumor type. In one embodiment, the fourth predetermined criteria includes a match of the gene variant and patient tumor type to the annotation of the fourth object.

In certain embodiments, the one or more processors may further apply the number of rulesets against the set of genomic and medical data by comparing the set of genomic and medical data against a respective ruleset corresponding to clinical trials, and determining whether the set of genomic and medical data satisfies a fifth predetermined criteria based on the clinical trials. In one embodiment, the fifth predetermined criteria includes a sensitivity to a targeted therapy type. In certain embodiments, based on a determination that the set of genomic and medical data satisfies the fifth predetermined criteria, the one or more processors may display clinical trial options in a fifth predefined space of the predefined area. In certain embodiments, based on a determination that the set of genomic and medical data fails to satisfy the fifth predetermined criteria, the one or more processors may forgo displaying the clinical trial options in the fifth predefined space. In certain embodiments, the one or more processors may generate the fifth object in the knowledge base, in which the fifth object includes an annotation indicative of a gene variant, a tumor type, and a clinical trial patient recruitment criteria. In one embodiment, the fifth predetermined criteria includes a match of the gene variant and patient tumor type to the annotation of the fifth object.

In certain embodiments, the one or more processors may further apply the number of rulesets against the set of genomic and medical data by comparing the set of genomic and medical data against a respective ruleset corresponding to genomic findings with prognostic implications, and determining whether the set of genomic and medical data satisfies a sixth predetermined criteria based on the genomic findings with prognostic implications. In one embodiment, the sixth predetermined criteria includes a match of a gene variant and a patient tumor type to a sixth object in the knowledge base corresponding to the genomic findings with prognostic implications. In certain embodiments, based on a determination that the set of genomic and medical data satisfies the sixth predetermined criteria, the one or more processors may display the genomic findings with prognostic implications in a sixth predefined space of the predefined area. In certain embodiments, based on a determination that the set of genomic and medical data fails to satisfy the sixth predetermined criteria, the one or more processors may forgo displaying the genomic findings with prognostic implications in the sixth predefined space. In certain embodiments, the one or more processors may generate the sixth object in the knowledge base, in which the sixth object includes an annotation indicative of a gene variant and a tumor type. In one embodiment, the sixth predetermined criteria includes a match of the gene variant and patient tumor type to the annotation of the sixth object.

In certain embodiments, the one or more processors may further apply the number of rulesets against the set of genomic and medical data by comparing the set of genomic and medical data against a respective ruleset corresponding to genomic findings with germline implications, and determining whether the set of genomic and medical data satisfies a seventh predetermined criteria based on the genomic findings with germline implications. In certain embodiments, the seventh predetermined criteria includes: 1) a match of a germline mutation associated with one or more hereditary cancer predisposition syndromes to a seventh object in the knowledge base corresponding to the genomic findings with germline implications, and 2) a determination that the germline mutation is above a predetermined variant allele frequency (VAF) threshold. In certain embodiments, based on a determination that the set of genomic and medical data satisfies the seventh predetermined criteria, the one or more processors may display the genomic findings with germline implications in a seventh predefined space of the predefined area. In certain embodiments, based on a determination that the set of genomic and medical data fails to satisfy the seventh predetermined criteria, the one or more processors may forgo displaying the genomic findings with germline implications in the seventh predefined space. In certain embodiments, the one or more processors may generate the seventh object in the knowledge base, in which the seventh object includes an annotation indicative of a gene variant and a tumor type. In one embodiment, the seventh predetermined criteria comprises a match of the germline mutation to the annotation of the seventh object.

In certain embodiments, the one or more processors may further apply the number of rulesets against the genomic testing data by comparing the set of genomic and medical data against a respective ruleset corresponding to genomic findings with clonal hematopoiesis (CH) implications, and determining whether the set of genomic and medical data satisfies an eighth predetermined criteria based on the genomic findings with CH implications. In certain embodiments, the eighth predetermined criteria includes: 1) a match of a CH to an eighth object in the knowledge base corresponding to the genomic findings with CH implications, and 2) a determination that the CH is associated with one or more particular analytes. In certain embodiments, based on a determination that the set of genomic and medical data satisfies the eighth predetermined criteria, the one or more processors may display the genomic findings with CH implications in an eighth predefined space of the predefined area. In certain embodiments, based on a determination that the set of genomic and medical data fails to satisfy the eighth predetermined criteria, the one or more processors may forgo displaying the genomic findings with CH implications in the eighth predefined space. In certain embodiments, the one or more processors may generate the eighth object in the knowledge base, in which the eighth object includes an annotation indicative of a gene variant and a tumor type. In one embodiment, the eighth predetermined criteria includes a match of the CH to the annotation of the eighth object.

In certain embodiments, the one or more processors may cause an electronic device to display the report by causing the electronic device to display the report including the predefined area for the one or more natural-language phrases on a first page of the report. In certain embodiments, the one or more natural-language phrases may include an embedded hyperlink associated with the one or more natural-language phrases. In certain embodiments, the embedded hyperlink may include a link to one or more other pages of the report, in which the one or more other pages include a set of detailed information corresponding to the one or more natural-language phrases. In certain embodiments, the embedded hyperlink may include a page number of the one or more other pages. In certain embodiments, the one or more processors may generate the report by generating a companion diagnostic (CDx) report associated with the patient. In certain embodiments, the one or more processors may cause an electronic device to display the report by causing a human machine interface (HMI) associated with a clinician to display the report. In certain embodiments, the one or more processors may cause an electronic device to display the report by causing an electronic device associated with the patient to display the report. In certain embodiments, the one or more processors may further cause the electronic device to display the report including the one or more natural-language phrases in a second predefined area. In certain embodiments, the second predefined area includes an appendix on one or more rearward pages of the report. In certain embodiments, the appendix may include the one or more natural-language phrases displayed along with additional contextual information, such as information that may be determined to be relevant to the patient.

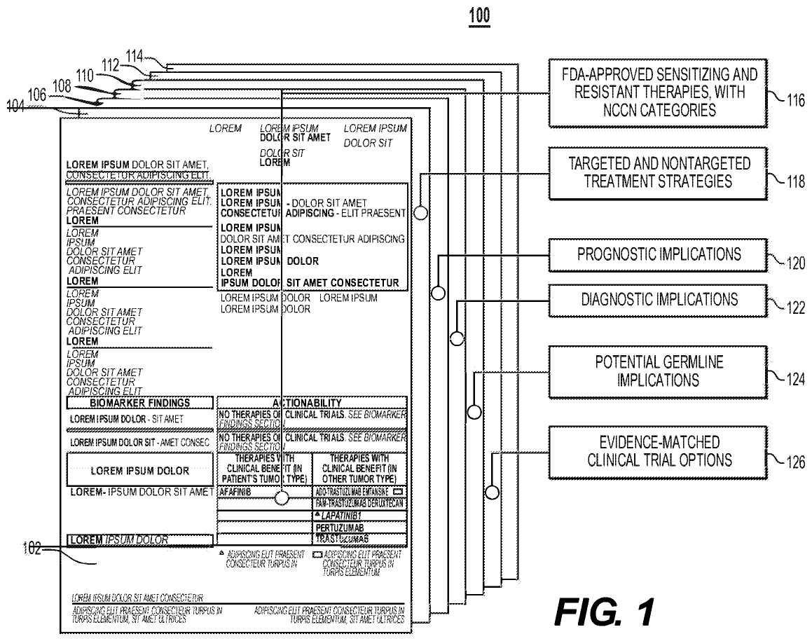

FIG. 1 provides a non-limiting example of a genomic sequencing clinical report 100. In certain embodiments, the genomic sequencing clinical report 100 may include a number of pages 102, 104, 106, 108, 110, 112, and 114. As depicted, in some embodiments, the number of pages 102, 104, 106, 108, 110, 112, and 114 may include, for example, genomic and medical information (e.g., FDA-approved sensitizing and resistant therapies 116, targeted and non-targeted treatment strategies, prognostic implications 120, diagnostic implications 122, potential germline implications 124, evidence-matched clinical trials options 126, and so forth) that may be dispersed across various and differing pages of the number of pages 102, 104, 106, 108, 110, 112, and 114. However, because the genomic and medical information (e.g., FDA-approved sensitizing and resistant therapies 116, targeted and non-targeted treatment strategies, prognostic implications 120, diagnostic implications 122, potential germline implications 124, evidence-matched clinical trials options 126, and so forth) dispersed across various and differing pages 102, 104, 106, 108, 110, 112, and 114 may often be presented within the clinical report in a counterintuitive and/or deemphasized manner, clinicians and patients alike may struggle to distinguish genomic and medical information of high clinical import from that of less clinical import to the patient. This may lead to clinicians and/or patients overlooking clinically significant information within the genomic sequencing clinical report 100, and potentially decreasing the overall efficacy of care provided to patients.

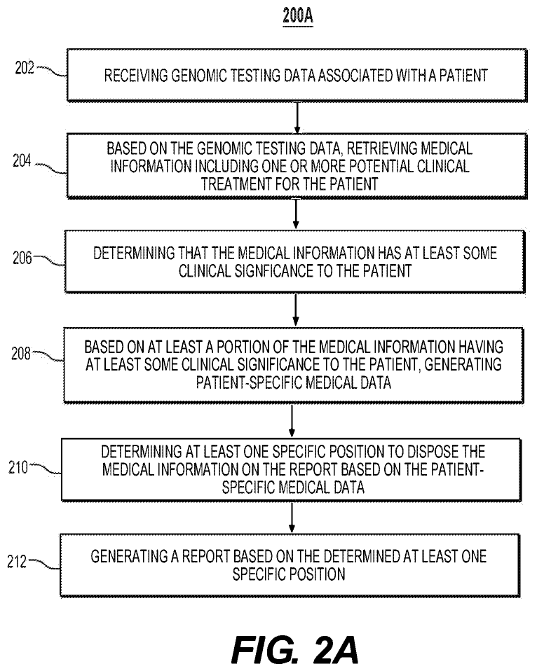

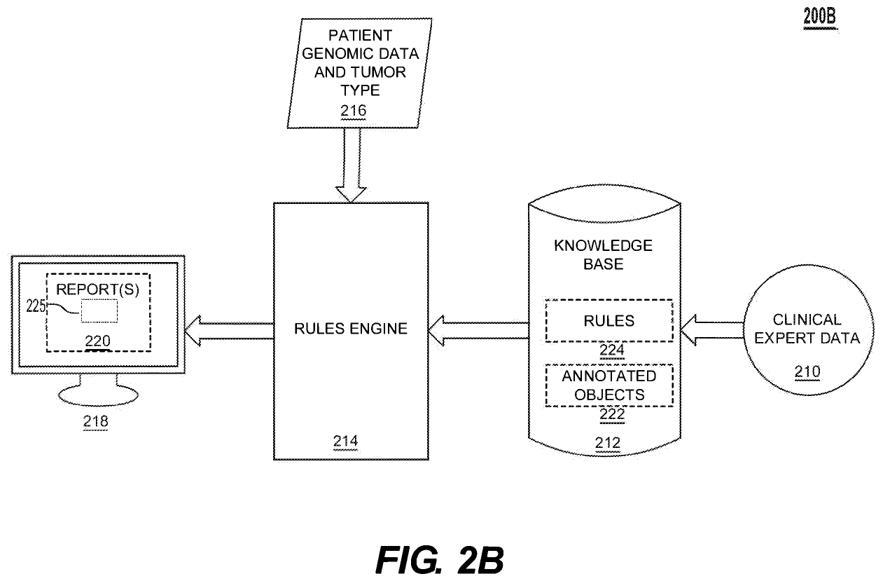

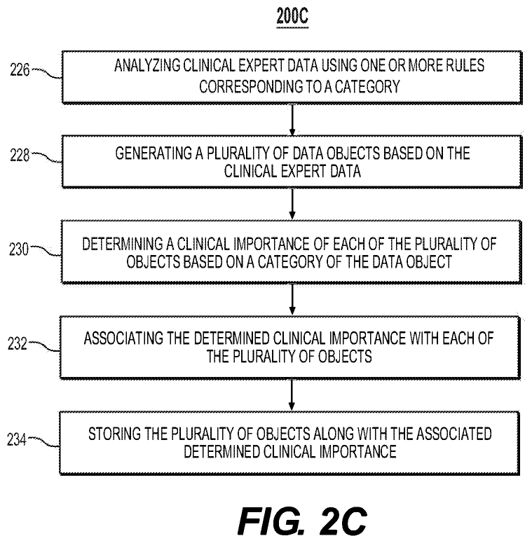

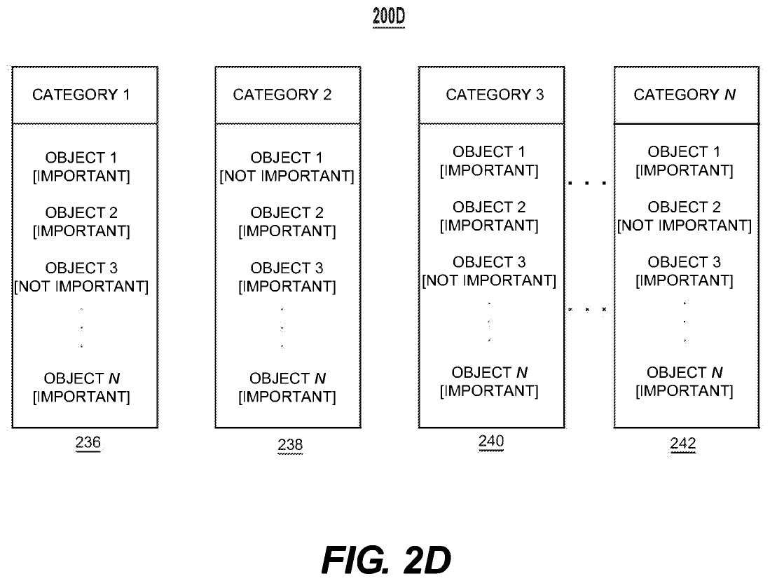

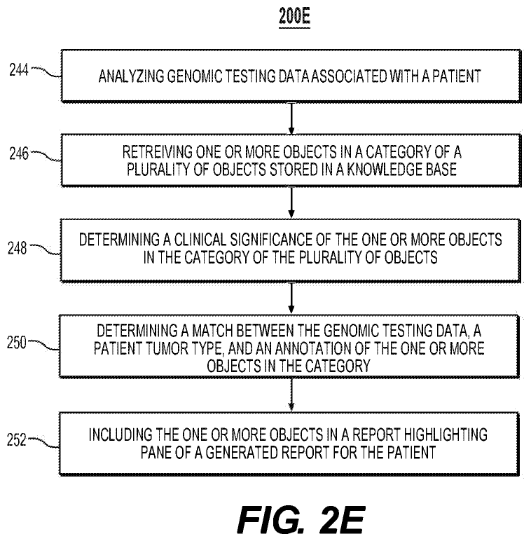

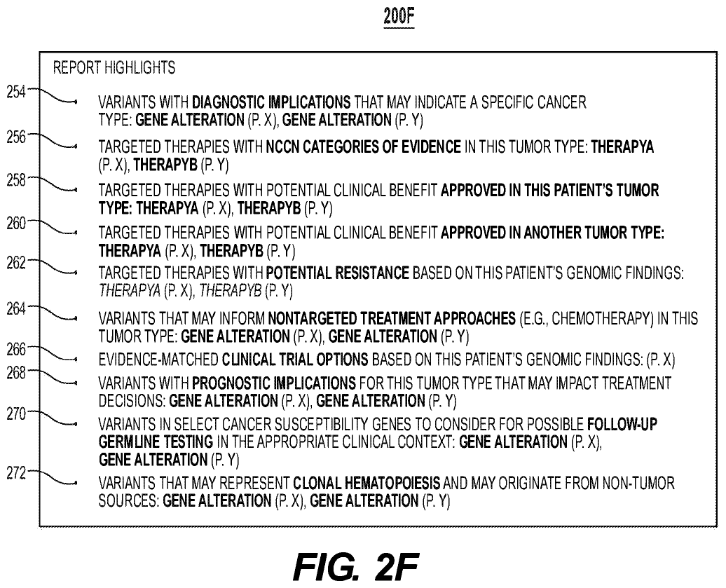

FIG. 2A provides a non-limiting example of a flow diagram of a process 200A for generating a report of genomic and medical information associated with a patient. Process 200A can be performed, for example, using one or more electronic devices implementing a software platform. In some examples, process 200A is performed using a client-server system, and the blocks of process 200A are divided up in any manner between the server and a client device. In other examples, the blocks of process 200A are divided up between the server and multiple client devices. Thus, while portions of process 200A are described herein as being performed by particular devices of a client-server system, it will be appreciated that process 200A is not so limited. In other examples, process 200A is performed using only a client device or only multiple client devices. In process 200A, some blocks are, optionally, combined, the order of some blocks is, optionally, changed, and some blocks are, optionally, omitted. In some examples, additional steps may be performed in combination with the process 200A. Accordingly, the operations as illustrated (and described in greater detail below) are exemplary by nature and, as such, should not be viewed as limiting.

The process 200A may begin at block 202 with the one or more electronic devices receiving genomic testing data associated with the patient. The process 200A may then continue at block 204 with the one or more electronic devices, based on the genomic testing data, retrieving medical information including one or more potential clinical treatments for the patient. For example, in certain embodiments, the medical data may correspond to one or more annotated data objects associating genomic findings and tumor type with one or more therapeutics or treatments that may be retrieved from a knowledge base based on a patient's genomic testing data. The process 200A may then continue at block 206 with the one or more electronic devices determining that the medical information has at least some clinical significance to the patient. For example, in certain embodiments, the patient-specific medical data may include an annotation of the one or more annotated data objects that may be retrieved from the knowledge base and determined to be clinically significant to the patient. The annotation may be presented within a report highlighting pane on, for example, a first page of the report. The process 200A may then continue at block 208 with the one or more electronic devices, based on at least a portion of the medical information having at least some clinical significance to the patient, generating patient-specific medical data. The process 200A may then continue at block 210 with the one or more electronic devices determining at least one specific position to dispose the medical information on the report based on the patient-specific medical data. For example, in certain embodiments, the specific position and/or specific location may include, for example, the order and placement of information on the first page of the report. The process 200A may then continue at block 212 with the one or more electronic devices generating, by the one or more processors, the report based on the determined specific position. For example, in certain embodiments, a report of genomic and medical information associated with a patient may be generated, in which the report automatically highlights and emphasizes the most clinically significant genomic and medical information. This may allow clinicians and patients to more readily ascertain genomic and medical information having the highest impact to patient care, and, by extension, improve the overall efficacy of care provided to patients.

FIG. 2B illustrates a non-limiting example of a computing architecture 200B for generating a report of genomic and medical information associated with a patient. In certain embodiments, the computing architecture 200B may include clinical expert data 210, a knowledge base 212, a rules engine 214, patient genomic data and patient tumor type 216, and electronic device 218 that may display a report 220. It should be appreciated that the computing architecture 200B may represent only an embodiment of a computing architecture that may be suitable for generating a report of genomic and medical information associated with a patient, in accordance with the presently disclosed techniques. For example, in certain embodiments, the computing architecture 200B may, in addition to, or in place of the rules engine 214, include one or more machine-learning models (e.g., supervised machine-learning model, unsupervised machine-learning model, deep learning model, and so forth) that may be utilized to receive the clinical expert data 210 from the knowledge base 212 and output one or more predicted rules or associations based on the clinical expert data 210 and to be applied to the patient genomic data and patient tumor type 216.

In certain embodiments, the clinical expert data 210 may include, for example, scientific, medical, and/or clinical research literature that may be inputted to the knowledge base 212 (e.g., over time). For example, in certain embodiments, the clinical expert data 210 may include, for example, scientific, medical, and clinical research literature and data including National Comprehensive Cancer Network (NCCN) Guidelines, European Society of Medical Oncology (ESMO) Guidelines, World Health Organization (WHO) Guidelines, Association of Molecular Pathology (AMP) Guidelines, College of American Pathologists (CAP) Guidelines, consensus recommendations by well-powered clinical studies published in peer-reviewed publications, professional conference abstracts, medical library references, and so forth. In certain embodiments, the knowledge base 212 may include, for example, one or more databases that may be suitable for storing annotated data objects 222 (e.g., one or more data structures or modules associating certain genomic findings and/or gene variants with a clinical significance and one or more therapies or treatments for a given tumor type) and rules 224 that may be utilized by the rules engine 214. In certain embodiments, the knowledge base 212 may be constantly updated with the clinical expert data 210.

In certain embodiments, the rules engine 214 may include, for example, a software system, a hardware system, a combination of software and hardware, programmable logic, an expert system, or other system or subsystem that may be suitable for receiving the patient genomic data and patient tumor type 216 and retrieving one or more rules 224 from the knowledge base 212 to apply against the patient genomic data and patient tumor type 216. For example, in certain embodiments, the rules engine 214 may execute one or more of the rules 224 on the patient genomic data and patient tumor type 216, and, if any condition (e.g., an annotation of one or more of the annotated data objects 222 corresponding to a particular gene variant or combination of variants, a clinically significant category, a particular tumor type, a particular therapy, a particular treatment, and so forth) matches, executes an associated action. In certain embodiments, the rules engine 214 may execute the associated action by generating one or more reports 220 (e.g., NGS reports) to be displayed on one or more computing devices 218. For example, in certain embodiments, the one or more reports 220 may include a report highlighting pane 225 (e.g., a predefined area including a number of predefined spaces) for prominently displaying the retrieved subset of one or more annotated objects, in accordance with the presently disclosed techniques. In this way, as will be further appreciated below, the computing architecture 200B may thus generate one or more reports 220 of genomic and medical information associated with a patient, in which the report automatically highlights and emphasizes the most clinically significant genomic and medical information by including the most clinically significant genomic and medical information in a report highlighting pane 225 on the first page of the report 220. This may thus allow clinicians and patients to more readily ascertain genomic and medical information having the highest impact to patient care, and, by extension, improve the overall efficacy of care provided to patients.

FIG. 2C provides a non-limiting example of a flow diagram of a process 200C for generating and storing a number of annotated data objects to a knowledge base, and FIG. 2D illustrates an example embodiment of a knowledge base 200D that may include a predetermined set of categories and annotated data objects, in accordance with the presently disclosed embodiments. Process 200C can be performed, for example, using one or more electronic devices implementing a software platform. In some examples, process 200C is performed using a client-server system, and the blocks of process 200C are divided up in any manner between the server and a client device. In other examples, the blocks of process 200C are divided up between the server and multiple client devices. Thus, while portions of process 200C are described herein as being performed by particular devices of a client-server system, it will be appreciated that process 200C is not so limited. In other examples, process 200 is performed using only a client device or only multiple client devices. In process 200C, some blocks are, optionally, combined, the order of some blocks is, optionally, changed, and some blocks are, optionally, omitted. In some examples, additional steps may be performed in combination with the process 200C.

For example, in certain embodiments, the process 200C may performed for each of a predetermined set of clinically significant genomic or therapeutic categories, including, for example, a genomic findings with diagnostic implications category, a targeted therapies with highest level of evidence category, a targeted therapies with expected resistance category, a genomic findings with non-targeted therapy implications category, an evidence-matched clinical trial options category, a genomic findings with prognostic implications category, a genomic findings with germline implications category, a genomic findings with clonal hematopoiesis (CH) implications category, a genomic findings with companion diagnostic (CDx) category, a targeted therapies with pan-tumor indications category, or an investigational targeted therapies category. In accordance with the presently disclosed embodiments, the process 200C may be performed iteratively for each of the aforementioned categories to generate and store a number of annotated data objects to a knowledge base. For example, in certain embodiments, referring to FIG. 2D, category 236 (e.g., “Category 1”), category 238 (e.g., “Category 2”), category 240 (e.g., “Category 3”), and category 242 (e.g., “Category N”) may each correspond to a different one of the genomic findings with diagnostic implications category, targeted therapies with highest level of evidence category, targeted therapies with expected resistance category, genomic findings with non-targeted therapy implications category, an evidence-matched clinical trial options category, genomic findings with prognostic implications category, a genomic findings with germline implications category, genomic findings with CH implications category, and so forth. As depicted, the category 236 (e.g., “Category 1”), category 238 (e.g., “Category 2”), category 240 (e.g., “Category 3”), and category 242 (e.g., “Category N”) may include a number of annotated data objects that may be further indicated being clinically significant or not clinically significant. Accordingly, the operations as illustrated (and described in greater detail below) are exemplary by nature and, as such, should not be viewed as limiting.

The process 200C may begin at block 226 with the one or more electronic devices analyzing clinical expert data utilizing one or more corresponding to a category. The process 200C may then continue at block 228 with the one or more electronic devices generating a plurality of data objects based on the clinical expert data. The process 200C may then continue at block 230 with the one or more electronic devices determining a clinical importance of each of the number of data objects based on the category of the data object. The process 200C may then continue at block 232 with the one or more electronic devices associating the determined clinical importance with each of the number of data objects. The process 200C may then conclude at block 234 with the one or more electronic devices storing the number of objects along with the associated determined clinical importance.

For example, in certain embodiments, for the genomic findings with diagnostic implications category, generating and storing a number of annotated data objects 222 to the knowledge base 212 may include analyzing information in the clinical expert data 210 to be generated for reports 220. For example, the sources may include peer-reviewed published articles, conference abstracts, professional guidelines, and publicly available databases, and so forth. In certain embodiments, information identified that indicates a specific genomic finding may be diagnostic of or highly prevalent in a specific tumor type may prompt creation of a first data object (e.g., one or more data objects included as part of predetermined categories 236, 238, 240, 242) within the knowledge base 212, including descriptive text annotated both to specific classes of gene alterations and to specific tumor types. For example, in some embodiments, the object includes a first data item indicating one or more gene variants and one or more tumor types (e.g., cancer tumors) associated with diagnostic implications, and a second data item including descriptive text and reference citations. In block 230, the system determines a clinical importance for the data object. For example, if this information also meets a predetermined criteria for inclusion in the report highlighting pane 225, which may include presence in NCCN or ESMO treatment algorithms, recommendation by AMP, CAP, or WHO, consensus recommendation by NCCN, and/or consensus recommendation by well-powered published clinical studies, then a third data item (e.g., selection of a specific checkbox) is annotated on the first data object for eligibility to be included in the report highlighting pane 225. The clinical expert data 210 may be continuously monitored for information updates through mechanisms including surveillance of journal publications, review of oncology conference abstracts, review of new or updated professional guidelines, and so forth.

In certain embodiments, for the targeted therapies with highest level of evidence category, generating and storing a number of annotated data objects 222 to the knowledge base 212 may include analyzing information in the clinical expert data 210 to be generated for reports 220. For example, if there is information in the clinical expert data 210 that a genomic finding is associated with sensitivity to a molecularly targeted therapy that is approved by a regulatory agency for one or more oncology indications, then a second data object (e.g., one or more data objects included as part of predetermined categories 236, 238, 240, 242) may be generated in the knowledge base 214, including the name of the specific therapy annotated both to specific classes of gene alterations and to specific tumor types. For example, in some embodiments, the object includes a first data item indicating one or more gene variants and one or more tumor types (e.g., cancer tumors) associated with sensitivity to the specific therapy, a second data item indicating NCCN categories of evidence (e.g., 1, 2A, 2B, 3) associated with the specific therapy for one or more tumor types, a third data item indicating a regulatory agency (e.g., FDA, EMA, others) and one or more tumor types for which the specific therapy would be considered to be approved In Tumor Type (ITT), and a fourth data item indicating a regulatory agency (e.g., FDA, EMA, others) and one or more tumor types for which the specific therapy would be considered to be approved in Other Tumor Type (OTT). In certain embodiments, information identified in the clinical expert data 210 that indicates presence of a specific genomic finding may be associated with sensitivity to a specific targeted therapy may prompt creation of the second data object within the knowledge base 212. If this information also meets a predetermined criteria for inclusion in the report highlighting pane 225, which may include either having an NCCN category of evidence, a designation of approved ITT, or a designation of approved OTT for a specific combination of a gene variant and a tumor type, then the second data object is eligible to be included in the report highlighting pane 225. The clinical expert data 210 may be continuously monitored for information updates through mechanisms including review of new or updated regulatory agency approvals of targeted therapies for oncology indications, surveillance of journal publications, review of oncology conference abstracts, review of new or updated professional guidelines, and so forth.

In certain embodiments, for the targeted therapies with expected resistance category, generating and storing a number of annotated data objects 222 to the knowledge base 212 may include analyzing information in the clinical expert data 210 to be generated for reports 220. For example, if there is information in the clinical expert data 210 that a genomic finding is associated with expected resistance to a specific molecularly targeted therapy then a third data object (e.g., one or more data objects included as part of predetermined categories 236, 238, 240, 242) may be generated within the knowledge base 212, including the name of the specific therapy annotated both to specific classes of gene alterations and to specific tumor types. For example, in some embodiments, the object includes a first data item indicating one or more gene variants and one or more tumor types (e.g., cancer tumors) associated with the specific targeted therapy and a second data item indicating the level of expected resistance (e.g., full resistance, unclear resistance, or sensitive) based on the strength of information in the clinical expert data 210. Information considered in the clinical expert data 210 may include regulatory approval labels for the therapy (e.g., FDA, EMA, others), presence in professional guidelines (e.g., NCCN, ESMO, others), presence in published clinical studies or conference abstracts, and so forth. If this information also meets a predetermined criteria for inclusion in the report highlighting pane 225, which may for example be that the second data item is a specific level of resistance (e.g., full resistance), then the third data object may be eligible to be included in the report highlighting pane 225. The clinical expert data 210 may be continuously monitored for information updates through mechanisms including surveillance of journal publications, review of oncology conference abstracts, review of new or updated professional guidelines, and so forth.

In certain embodiments, for the genomic findings with non-targeted therapy category, generating and storing a number of annotated data objects 222 to the knowledge base 212 may include analyzing information in the clinical expert data 210 to be generated for reports 220. For example, determining that there is information in the clinical expert data 210 that a genomic finding is associated with improved or inferior outcomes with non-targeted treatment approaches (e.g., chemotherapy, radiotherapy, stem cell transplant, others) for patients with a specific tumor type may prompt creation of a fourth data object (e.g., one or more data objects included as part of predetermined categories 236, 238, 240, 242) within the knowledge base 212, including descriptive text annotated both to specific classes of gene alterations and to specific tumor types. For example, in some embodiments, the object includes a first data item indicating one or more gene variants and one or more tumor types (e.g., cancer tumors) associated with one or more non-targeted treatment approaches, and a second data item including descriptive text and citations. If this information also meets a predetermined criteria for inclusion in the report highlighting pane 225, which may include a presence in NCCN or ESMO treatment algorithms, recommendation by AMP, CAP, or WHO, consensus recommendation by NCCN, and/or consensus recommendation by well-powered published clinical studies, then a third data item (e.g., selection of a specific checkbox) is annotated on the fourth data object for eligibility to be included in the report highlighting pane 225. The clinical expert data 210 may be continuously monitored for information updates through mechanisms including surveillance of journal publications, review of oncology conference abstracts, review of new or updated professional guidelines, and so forth.

In certain embodiments, for the genomic findings with prognostic implications category, generating and storing a number of annotated data objects 222 to the knowledge base 212 may include analyzing information in the clinical expert data 210 to be generated for reports 220. For example, determining that there is information in the clinical expert data 210 that a genomic finding is associated with prognostic implications (e.g., associated with improved or inferior prognosis or risk stratification) for patients with a specific tumor type may prompt creation of a sixth data object (e.g., one or more data objects included as part of predetermined categories 236, 238, 240, 242) within the knowledge base 212, including descriptive text annotated both to specific classes of gene alterations and to specific tumor types. For example, in some embodiments, the object includes a first data item indicating a gene variant and a type of tumor (e.g., a cancer tumor) associated with prognostic implications, and a second data item including descriptive text and citations. If this information also meets a predetermined criteria for inclusion in the report highlighting pane 225, which may include a presence in NCCN or ESMO treatment algorithms, recommendation by AMP, CAP, or WHO, consensus recommendation by NCCN, and/or consensus recommendation by well-powered published clinical studies, then a third data item (e.g., selection of a specific checkbox) is annotated on the sixth data object for eligibility to be included in the report highlighting pane 225. The clinical expert data 210 may be continuously monitored for information updates through mechanisms including surveillance of journal publications, review of oncology conference abstracts, review of new or updated professional guidelines, and so forth.

In certain embodiments, for the genomic findings with germline implications category, generating and storing a number of annotated data objects 222 to the knowledge base 212 may include analyzing information in the clinical expert data 210 to be generated for reports 220. For example, a list of clinically relevant genes with potential germline implications (cancer susceptibility genes) is determined based on professional guidelines (e.g., ACMG, ESMO). Within these genes, variants that may represent a potential germline pathogenic mutation associated with hereditary cancer pre-disposition syndromes, which may include a specific variants included in the ClinVar genomic database meeting a specific significance level (e.g., Pathogenic, Pathogenic/Likely Pathogenic, Likely Pathogenic) supported by more than one submitter, may prompt creation of a seventh data object (e.g., one or more data objects included as part of predetermined categories 236, 238, 240, 242) including descriptive text annotated both to specific classes of gene alterations and to specific tumor types. For example, in some embodiments, the object includes a first data item indicating one or more gene variant(s) and one or more tumor type(s) (e.g., cancer tumors) associated with potential germline implications, and a second data item including descriptive text and citations. In certain embodiments, if this information also satisfies a predetermined criteria, which may include the subset of ClinVar variants that occur in specific clinically actionable genes based on ESMO recommendation with a ≥10% probability of germline origin when identified during tumor sequencing, then a third data item (e.g., selection of a specific checkbox) is annotated on the seventh data object for eligibility to be included in the report highlighting pane 225. In some embodiments, analysis of internal VAF data observed for a set of common germline variants may be utilized to determine and include particular assay-specific VAF thresholds which must be met for final inclusion in the report highlighting pane 225. The clinical expert data 210 may be continuously monitored for information updates through mechanisms including review of new or updated entries in the ClinVar database, surveillance of journal publications, review of oncology conference abstracts, review of new or updated professional guidelines, and so forth.

In certain embodiments, for the genomic findings with CH implications category, generating and storing a number of annotated data objects 222 to the knowledge base 212 may include analyzing information in the clinical expert data 210 to be generated for reports 220. For example, determining that there is information in the clinical expert data 210 that a genomic finding is associated with CH implications with respect to a specific tumor type may prompt creation of an eighth data object (e.g., one or more data objects included as part of predetermined categories 236, 238, 240, 242) within the knowledge base 212, including descriptive text annotated both to specific classes of gene alterations and to specific tumor types. For example, in some embodiments, the object includes a first data item indicating one or more gene variant(s) and one or more tumor type(s) (e.g., cancer tumors) associated with potential CH implications, and a second data item including descriptive text and citations. If this information also satisfies a predetermined criteria, which may include a consensus recommendation by well-powered published clinical studies, then a third data item (e.g., selection of a specific checkbox) is annotated on the eighth data object for eligibility to be included in the report highlighting pane 225. If relevant, additional rules associated with the eighth data object may specify that the eighth data object may only be active for certain NGS sample types for final inclusion in the report highlighting pane 225. The clinical expert data 210 may be continuously monitored for information updates through mechanisms including surveillance of journal publications, review of oncology conference abstracts, review of new or updated professional guidelines, and so forth.

FIG. 2E provides a non-limiting example of a flow diagram of a process 200E populating a report highlighting pane of a report associated with a patient with one or more annotated data objects. Process 200E can be performed, for example, using one or more electronic devices implementing a software platform. In some examples, process 200E is performed using a client-server system, and the blocks of process 200E are divided up in any manner between the server and a client device. In other examples, the blocks of process 200E are divided up between the server and multiple client devices. Thus, while portions of process 200E are described herein as being performed by particular devices of a client-server system, it will be appreciated that process 200E is not so limited. In other examples, process 200E is performed using only a client device or only multiple client devices. In process 200E, some blocks are, optionally, combined, the order of some blocks is, optionally, changed, and some blocks are, optionally, omitted. In some examples, additional steps may be performed in combination with the process 200E. For example, in certain embodiments, the process 200E may performed for each of a predetermined set of clinically significant] genomic or therapeutic categories, including, for example, a genomic findings with diagnostic implications category, a targeted therapies with highest level of evidence category, a targeted therapies with expected resistance category, a genomic findings with non-targeted therapy implications category, an evidence-matched clinical trial options category, a genomic findings with prognostic implications category, a genomic findings with germline implications category, a genomic findings with clonal hematopoiesis (CH) implications category, a genomic findings with companion diagnostic (CDx) category, a targeted therapies with pan-tumor indications category, or an investigational targeted therapies category. Accordingly, the operations as illustrated (and described in greater detail below) are exemplary by nature and, as such, should not be viewed as limiting.

The process 200E may begin at block 244 with the one or more electronic devices analyzing genomic testing data associated with a patient. The process 200E may then continue at block 246 with the one or more electronic devices retrieving one or more data objects in a category of a number of data objects stored to a knowledge base. The process 200E may then continue at block 248 with the one or more electronic devices determining a clinical significance of the one or more data objects in the category of the number of data objects. The process 200E may then continue at block 250 with the one or more electronic devices determining a match between the genomic testing data, patient tumor type, and annotation of the one or more data objects in the category. The process 200E may then conclude at block 252 with the one or more electronic devices including the one or more data object in a report highlighting pane of generated report for a patient.

In certain embodiments, the rules engine 214 may then apply the rules 224 against the patient genomic data and patient tumor type 216. For example, in certain embodiments, if a patient's genomic sequencing clinical report 220 is being generated and the patient's tumor type and genomic findings (e.g., gene variant) match the annotation of the first data object, then the first data object may be pulled into the patient's report and displayed in the text for that gene under the “Potential Diagnostic Implications” header in the “Genomic Findings” section 315 of the report. Further, if the first data object has the specific checkbox selected, then the genomic findings with diagnostic implications category 254 highlight will also appear in the report highlighting pane 225 on the first page of the genomic sequencing clinical report 220 as a bullet point with the gene alteration and the page number linking to the “Potential Diagnostic Implications” header for that gene in the “Genomic Findings” section 315 on one or more other pages of the genomic sequencing clinical report 220. For example, in a simplex example, a gene variant may include an annotation “X.” Patient genomic findings may be compared to the annotation “X”, and if one or more patient genomic findings match the gene variant in the annotation “X”, the patient genomic findings may be determined as being clinically significant and the annotation “X” will be retrieved from the knowledge base for display along with the patient genomic findings in the report highlighting pane 225.

In certain embodiments, for the targeted therapies with highest level of evidence category 256, the sets of annotated data objects 222 and the rules 224 may proceed as follows: if there is information in the clinical expert data 210 that a genomic finding is associated with sensitivity to an approved molecularly targeted therapy, then a second data object may be generated in the knowledge base 212, which may include indications of NCCN categories of evidence for the therapy, tumor types for which the therapy would be considered approved In Tumor Type (ITT), and tumor types for which the therapy would be considered approved in Other Tumor Type (OTT), through annotations to specific classes of gene alterations and/or specific tumor types, thus rendering the therapy eligible to be included in the targeted therapies with highest level of evidence category 256 in the report highlighting pane 225.

In certain embodiments, the rules engine 214 may then apply the rules 224 against the patient genomic data and patient tumor type 216. For example, in certain embodiments, if a patient's genomic sequencing clinical report 220 is being generated and the patient's tumor type and genomic findings (e.g., gene variants) match the annotation of the second data object, then the second data object may be pulled into the patient's report and the name of the therapy may be displayed for that gene in the Actionability Table on the front page of the clinical report 220. Accompanying text for that therapy may be displayed in a section having a header, e.g., a “Therapies with Clinical Benefit” section of the report. If one or more of the second data objects are pulled into the patient's report, then the targeted therapies with highest level of evidence category 256 highlight will also appear in the report highlighting pane 225 on the first page of the genomic sequencing clinical report 220. If more than one of the second data objects are pulled into the patient's report, then only those with the highest level of evidence (e.g., prioritizing first therapies with an NCCN category, then therapies approved in the patient's tumor type [ITT], then therapies approved in other patient's tumor type [OTT]) may be presented in the report highlighting pane 225. The targeted therapies with highest level of evidence category 256 will appear in the report highlighting pane 225 as a bullet point with the therapy name or names and the page number linking to the section for each therapy in the “Therapies with Clinical Benefit” section on one or more other pages of the genomic sequencing clinical report 220.

In certain embodiments, the rules engine 214 may then apply the rules 224 against the patient genomic data and patient tumor type 216. For example, in certain embodiments, if a patient's genomic sequencing clinical report 220 is being generated and the patient's tumor type and genomic findings (e.g., gene variants) match the annotation of the third data object, and further if the third data object has an annotation indicating that the level of expected resistance is full resistance, then the third data object may be pulled into the patient's report and the name of the therapy may be displayed as resistant for that gene in a table, e.g., an Actionability Table on the front page of the clinical report 220. The accompanying text for that therapy may be displayed in the “Therapies Associated with Resistance” section of the report. If one or more of the third data objects are pulled into the patient's report, then the targeted therapies with expected resistance category 258 highlight will also appear in the report highlighting pane 225 on the first page of the genomic sequencing clinical report 220 as a bullet point with the therapy name or names and the page number linking to section for each therapy in the “Therapies Associated with Resistance” section on one or more other pages of the genomic sequencing clinical report 220.

In certain embodiments, the rules engine 214 may then apply the rules 224 against the patient genomic data and patient tumor type 216. For example, in certain embodiments, if a patient's genomic sequencing clinical report 220 is being generated and the patient's tumor type and genomic findings (e.g., gene variants) match the annotation of the fourth data object, then the fourth data object may be pulled into the patient's report and displayed in the text for that gene under the “Potential Treatment Strategies: Nontargeted Approaches” header in the “Genomic Findings” section 315 of the clinical report 220. Further, if the fourth data object has the specific checkbox selected, then the non-targeted therapy implications category 352 highlight will also appear in the report highlighting pane 225 on the first page of the genomic sequencing clinical report 220 as a bullet point with the gene variant and the page number linking to the “Potential Treatment Strategies: Nontargeted Approaches” header for that gene in the “Genomic Findings” section 315 on one or more other pages of the genomic sequencing clinical report 220.

As a non-limiting real-world example, presence of KMT2A (MLL) gene rearrangements are indicated in the NCCN Acute Lymphoblastic Leukemia (ALL) Guidelines as being a marker of reduced benefit from chemotherapy and improved outcomes from stem cell transplant for infants with high-risk ALL. A non-targeted treatment data object may be created in the knowledge base 212 describing this association and the box on this module is checked. The non-targeted treatment data object is annotated to all rearrangement alterations in the gene KMT2A (MLL) and to all internal tumor types classified as ALL or acute leukemia. When the report 220 is being generated for a patient with a tumor type of ‘B-lymphoblastic leukemia-lymphoma (B-ALL)’ harboring a KMT2A (MLL)-MLLT10 fusion, this specific non-targeted treatment data object will match. The non-targeted therapy implications will appear in the report highlighting pane 225 on the report front page as a bullet point listing the KMT2A (MLL)-MLLT10 fusion and the page number linking to the “Potential Treatment Strategies: Nontargeted Approaches” header, where the text from the annotation of the non-targeted treatment data object appears.

In certain embodiments, the rules engine 214 may then apply the rules 224 against the patient genomic data and patient tumor type 216. For example, in certain embodiments, if a patient's genomic sequencing clinical report 220 is being generated and the patient's tumor type, genomic findings (e.g., gene variants), and age match the annotation of the fifth data object, then the fifth data object may be pulled into the patient's report and displayed in association with that gene in the “Clinical Trials” section of the clinical report 220. If one or more of the fifth data objects are pulled into the patient's report, then the evidence-matched clinical trial options category 354 highlight will also appear in the report highlighting pane 225 on the first page of the genomic sequencing clinical report 220 as a bullet point with the page number linking to the “Clinical Trials” section on one or more other pages of the genomic sequencing clinical report 220.

As a non-limiting real-world example, presence of NRG1 gene fusions is reported in the scientific literature as being a potential marker of sensitivity to the investigational agent zenocutuzumab, a bi-specific antibody targeting HER2 and HER3. For example, NRG1 gene fusions promote oncogenesis by binding to and activating the HER3 receptor, and zenocutuzumab blocks this activation. Several patients with solid tumors harboring NRG1 fusion have benefitted from zenocutuzumab. A clinical trials central government database is queried for appropriate recruiting studies of zenocutuzumab, and NCT02912949 is identified. A clinical trial is created in MR2 and annotated to all rearrangements in the gene NRG1 and to all internal tumor types classified as solid tumors, and the age range is set to ≥18 years. When the report 220 is being generated for a patient aged 54 years with a tumor type of “Esophagus adenocarcinoma” harboring a CD74-NRG1 fusion, this specific clinical trial will match. The evidence-matched clinical trial options category 354 highlight will appear in the report highlighting pane 225 on the report front page as a bullet point listing the page number linking to the “Clinical Trials” section of the report, where the clinical trial NCT02912949 appears.

In certain embodiments, the rules engine 214 may then apply the rules 224 against the patient genomic data and patient tumor type 216. For example, in certain embodiments, if a patient's genomic sequencing clinical report 220 is being generated and the patient's tumor type and genomic findings match the annotation of the sixth data object, then the sixth data object may be pulled into the patient's report and displayed in the text for that gene under the “Frequency & Prognosis” header in the “Genomic Findings” section 315 of the clinical report 220. Further, if the sixth data object has the specific checkbox selected, then the genomic findings with prognostic implications category 356 highlight will also appear in the report highlighting pane 225 on the first page of the genomic sequencing clinical report 220 as a bullet point with the gene alteration or variant and the page number linking to the “Frequency & Prognosis” header for that gene in the “Genomic Findings” section 315 on one or more other pages of the genomic sequencing clinical report 220.

As a non-limiting real-world example, presence of KRAS gene mutations is indicated in the NCCN Non-Small Cell Lung Cancer (NSCLC) Guidelines as being a marker of inferior prognosis for patients with this type of lung cancer. A prognosis data object is created in the knowledge base 212 describing this association and the box on this module is checked. The prognosis data object is annotated to all activating mutations in the gene KRAS and to all internal tumor types classified as NSCLC. When the report 220 is being generated for a patient with a tumor type of ‘Lung adenocarcinoma’ harboring a KRAS G12V mutation, this specific prognosis data object will match. The prognostic implications will appear in the report highlighting pane on the report front page as a bullet point listing the KRAS G12V mutation and the page number linking to the “Frequency & Prognosis” header, where the text from the annotation of the prognosis data object appears.

In certain embodiments, the rules engine 214 may then apply the rules 224 against the patient genomic data and patient tumor type 216. For example, in certain embodiments, if a patient's genomic sequencing clinical report 220 is being generated and the patient's tumor type and genomic findings (e.g., gene variants) match the annotation of the seventh data object, then the seventh data object may be pulled into the patient's report and displayed in the text for that gene under the header “Potential Germline Implications” header 370 in the “Genomic Findings” section of the clinical report 220. Further, if the seventh data object has a specific checkbox selected and the genomic findings (e.g., gene variants) satisfies the VAF threshold criteria for the genomic test type set in the knowledge base 212, then the genomic findings with germline implications category 358 highlight will also appear in the report highlighting pane 225 on the first page of the genomic sequencing clinical report 220 as a bullet point with the gene alteration or variant and the page number linking to the “Potential Germline Implications” header 370 for that gene in the “Genomic Findings” section 315 on one or more other pages of the genomic sequencing clinical report 220.

As a non-limiting real-world example, MSH2 is a cancer susceptibility gene recommended in ACMG and ESMO guidelines for reporting of secondary germline findings and as having a ≥10% probability of germline origin when identified during tumor sequencing. The MSH2 A500fs*26 mutation is included in the ClinVar database as a germline variant of Pathogenic significance with Expert Panel review status and associated with Lynch syndrome. This mutation is therefore eligible to be highlighted as potential germline variant in report highlighting pane 225 if the assay-specific VAF threshold is met in the analyzed sample. A germline data object is created in the knowledge base 212 describing this association and the box on this germline data object is checked. The germline data object is annotated to all mutations in the gene MSH2 which have similar ClinVar status and Lynch syndrome association and to all available internal tumor types. An additional rule is set with respect the germline data object to specify that the germline data object checkbox is only active when the specified VAF threshold is met for each NGS test type (e.g., VAF ≥10% for FoundationOne CDx and FoundationOne Heme, VAF ≥30% for FoundationOne Liquid CDx). When the report 220 is being generated for a patient with a MSH2 A500fs*26 mutation at a VAF of 45%, this specific germline data object will match. The potential germline implications and a recommendation to consider follow-up germline testing will appear in the report highlighting pane 225 on the report front page as a bullet point listing the MSH2 A500fs*26 mutation and the page number linking to the “Potential Germline Implications” header, where the text from the germline data object appears.

In certain embodiments, the rules engine 214 may then apply the rules 224 against the patient genomic data and patient tumor type 216. For example, in certain embodiments, if a patient's genomic sequencing clinical report 220 is being generated and the patient's tumor type and genomic findings (e.g., gene variants) match the annotation of the eighth data object, then the eighth data object may be pulled into the patient's report and displayed in the text for that gene under the “Potential Clonal Hematopoiesis Implications” header in the “Genomic Findings” section 315 of the clinical report 220. Further, if the eighth data object also has a specific checkbox selected and the genomic test type matches criteria set in the knowledge base 212, then the genomic findings with CH implications category 360 highlight will also appear in the report highlighting pane 225 on the first page of the genomic sequencing clinical report 220 as a bullet point with the gene alteration or variant and the page number linking to the “Potential Clonal Hematopoiesis Implications” header for that gene in the “Genomic Findings” section 315 on one or more other pages of the genomic sequencing clinical report 220.

As a non-limiting real-world example, presence of ATM gene mutations is indicated in the scientific literature as potentially representative of CH when identified for patients with solid tumors by NGS analysis of a liquid biopsy. A CH data object is created in the knowledge base 212 describing this association and the box on this CH data object is checked. The CH data object is annotated to all short variant mutations in the gene ATM and to all internal tumor types classified as solid tumors. An additional rule is set with respect to the CH data object in which a checkbox is only active for certain types of NGS tests. When the report 220 is being generated for a patient with a tumor type of ‘Pancreas adenocarcinoma’ harboring an ATM Q2730P mutation, this CH data object will match. The CH implications highlight will appear in the report highlighting pane 225 on the report front page as a bullet point listing the ATM Q2730P mutation and the page number linking to the “Potential Clonal Hematopoiesis Implications” header, where the text from the CH data object appears.

FIG. 2F illustrates an example of a report highlighting pane 200F, including each category of an example predetermined set of treatment or therapeutic categories that may be included in the report highlighting pane 200F, in accordance with the presently disclosed embodiments. For example, in certain embodiments, the predetermined set of treatment or therapeutic categories of the report highlighting pane 200F may include, for example, a genomic findings with diagnostic implications category 254 (e.g., gene variants with diagnostic implications that may indicate a specific cancer type cancer type (e.g., “Gene Alteration (page X)” and “Gene Alteration (page Y)”)), a targeted therapies with highest level of evidence category (one of the three categories 256 (e.g., targeted therapies with NCCN categories of evidence for a particular tumor type (e.g., “Therapy A (page X)” and “Therapy B (page Y)”), 258 (e.g., targeted therapies with potential clinical benefit approved in the patient's tumor type (e.g., “Therapy A (page X)” and “Therapy B (page Y)”), or 260 (e.g., targeted therapies with potential clinical benefit approved in another tumor type other than the tumor type of the patient (e.g., “Therapy A (page X)” and “Therapy B (page Y)”)), a targeted therapies with expected resistance category 262 (e.g., targeted therapies with potential resistance based on the genomic findings for the particular patient (e.g., “Therapy A (page X)” and “Therapy B (page Y)”)), a genomic findings with non-targeted therapy implications category 264 (e.g., gene variants that may inform non-targeted approaches for the particular tumor type of the patient, such as chemotherapy (e.g., “Therapy A (page X)” and “Therapy B (page Y)”)), an evidence-matched clinical trial options category 266 (e.g., evidence-matched clinical trial options based on the patient's genomic findings (e.g., “(page X)”)), a genomic findings with prognostic implications category 268 (e.g., gene variants with prognostic implications for the particular tumor type that may impact treatment decisions (e.g., “Gene Alteration (page X)” and “Gene Alteration (page Y)”)), a genomic findings with potential germline implications category 270 (e.g., gene variants of select cancer susceptibility genes to consider for possible follow-up germline testing in the appropriate clinical context (e.g., “Gene Alteration (page X)” and “Gene Alteration (page Y)”)), and a genomic findings with clonal hematopoiesis (CH) implications category 272 (e.g., gene variants that may represent CH and may originate from non-tumor sources (e.g., “Gene Alteration (page X)” and “Gene Alteration (page Y)”)).

In other embodiments, the report highlighting pane 200F may further include, for example, additional categories, such as a genomic findings with companion diagnostic (CDx) category, a targeted therapies with pan-tumor indications category, or an investigational targeted therapies category. In certain embodiments, as previously discussed, the genomic findings with diagnostic implications category 254, the targeted therapies with highest level of evidence category 256, the targeted therapies with potential clinical benefit category 258, the targeted therapies with potential clinical benefit category 260, the targeted therapies with expected resistance category 262, the genomic findings with non-targeted therapy implications category 264, the evidence-matched clinical trial options category 266, the genomic findings with prognostic implications category 268, the genomic findings with germline implications category 270, and the genomic findings with CH implications category 272 may be presented, for example, in a bullet-pointed list according to a ranking from least clinically significant to most clinically significant to patient care, as depicted. In certain embodiments, the predetermined set of treatment or therapeutic categories may include natural-language phrases or annotations that may be learned and predicted by one or more machine-learning models (e.g., supervised machine-learning model, unsupervised machine-learning model, deep learning model, and so forth). In certain embodiments, the one or more machine-learning models (e.g., supervised machine-learning model, unsupervised machine-learning model, deep learning model, and so forth) may further learn and predict clinical significance of the data generated in the report. That is, the one or more machine-learning models may analyze all of the data of the report and/or data objects stored in the knowledge base to surface the clinically significant genomic data to patient care.

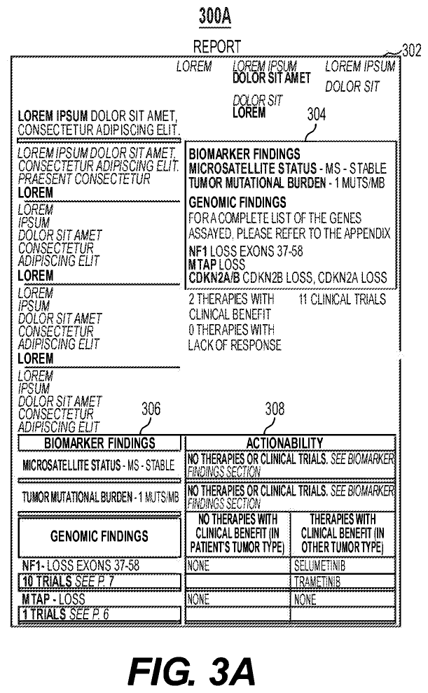

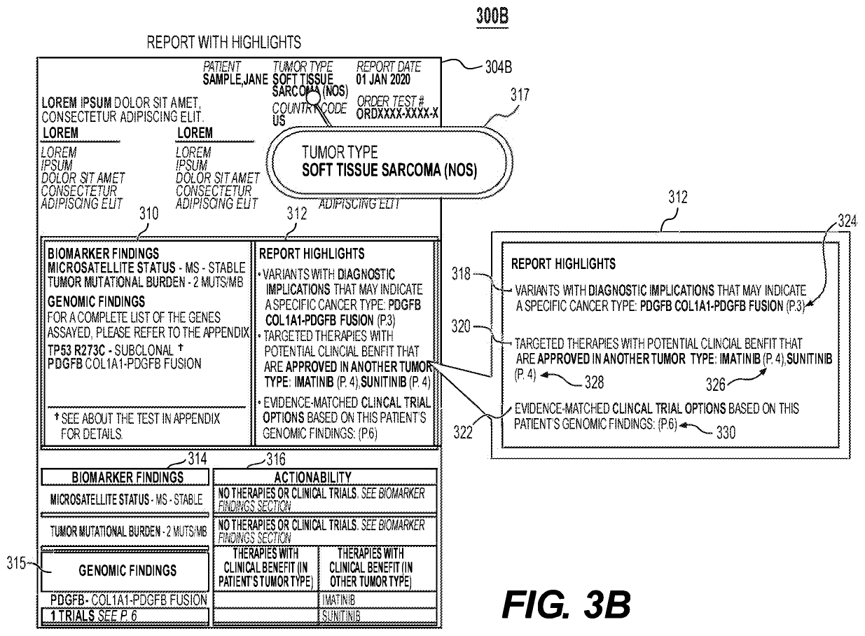

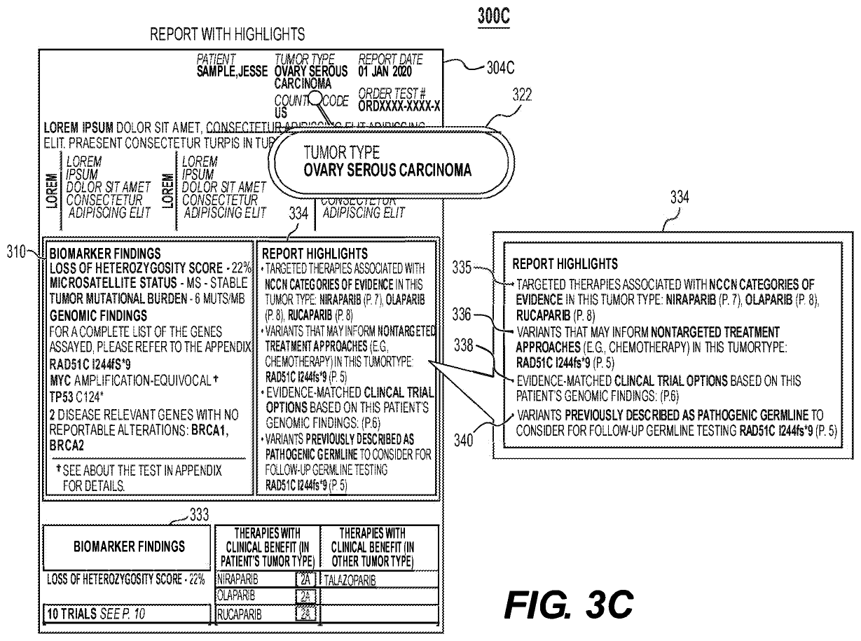

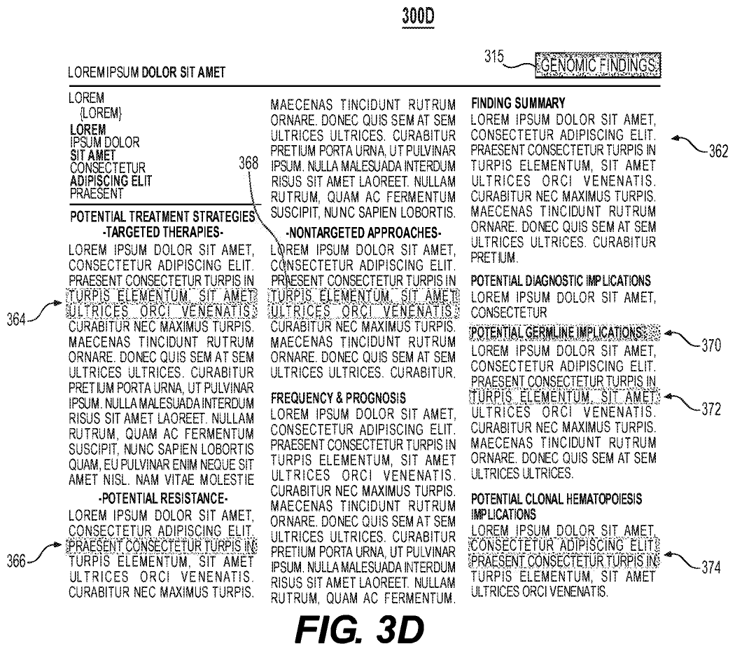

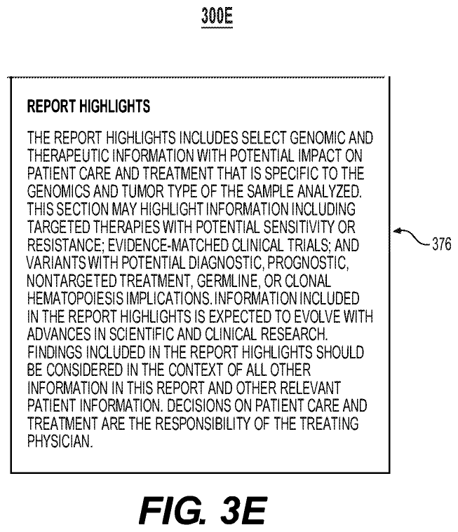

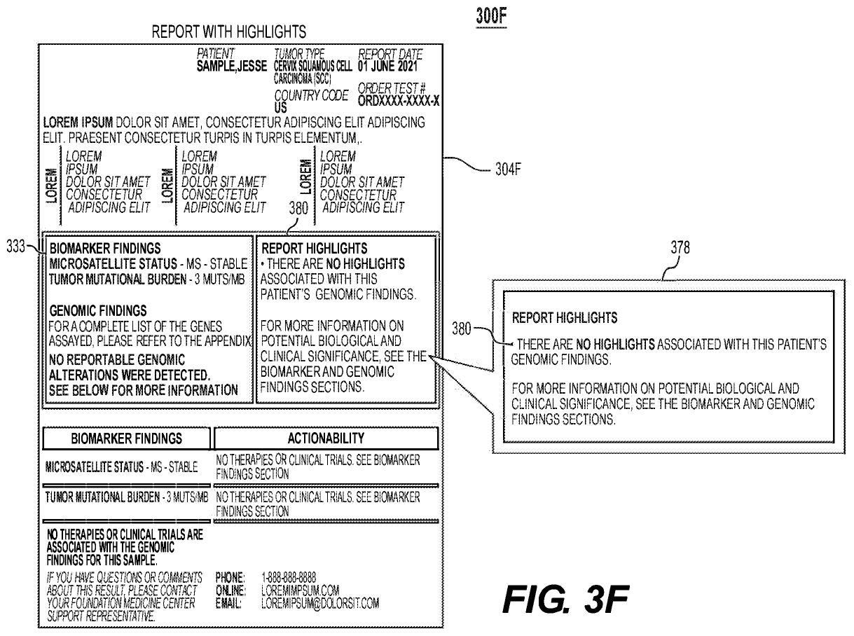

FIG. 3A provides a non-limiting example 300A of a genomic sequencing clinical report 302 without including a report highlighting pane. As depicted, the genomic sequencing clinical report 302 may include a biomarker and genomic findings summery pane 304, and an Actionability Table pane depicting biomarker and genomic findings 306 which are associated with sensitivity or resistance to molecularly targeted therapies that are approved for an oncology indication 308. As depicted, the genomic sequencing clinical report 302 that does not include a report highlighting pane prominently features biomarker and genomic findings pane 306 with implications for molecularly targeted therapies pane 308, and, although not illustrated, additional clinically significant and meaningful information (e.g., potential diagnostic or prognostic implications of the biomarker and genomic findings) may be presented, for example, on rearward pages of the genomic sequencing clinical report 302 (e.g., as opposed to prominently and neatly presenting all types of the most clinically significant and meaningful genomic and medical information on the first page of genomic sequencing clinical report 302).

FIGS. 3B and 3C provide non-limiting running examples of genomic sequencing clinical reports including a report highlighting pane. For example, as depicted by FIG. 3B, in contrast to the genomic sequencing clinical report 302 discussed above with respect to FIG. 3A, in certain embodiments, the genomic sequencing clinical report 304B may include a report highlighting pane 312 presented and displayed prominently on the first page of the genomic sequencing clinical report 304B. As depicted, in one embodiment, the report highlighting pane 312 may include, for example, genomic findings 318 (e.g., gene variants in the patient's genomic data includes diagnostic implications that may indicate a specific cancer type: PDGFB COL1A1-PDGFB fusion; genomic findings with prognostic implications; genomic findings with germline implications; genomic findings with CH implications; and so forth), targeted therapies 320 (e.g., targeted therapies with highest level of evidence; targeted therapies with expected resistance; and so forth), and evidence-matched clinical trial options 322 (e.g., evidence-matched clinical trial options that may be suitable to the particular patient based on the genomic data).

In certain embodiments, the genomic findings with diagnostic implications 318, targeted therapies with potential clinical benefit 320, and evidence-matched clinical trial options 322 may be presented, for example, in a bullet-pointed list of clinically relevant information. In certain embodiments, as further depicted, the genomic findings with diagnostic implications 318, targeted therapies with potential clinical benefit 320, and evidence-matched clinical trial options 322 may also be associated with embedded links 324, 326, 328, and 330 (e.g., corresponding to individual page numbers) that may be selected to link a patient, a clinician, or similar user to a landing page within the genomic sequencing clinical report 304B that includes a set of detailed information associated with the genomic findings with diagnostic implications 318, targeted therapies with potential clinical benefit 320, and evidence-matched clinical trial options 322. As further depicted, the genomic sequencing clinical report 304B may also include an actionability table including identified biomarker findings 314 or genomic findings 315 associated with potential sensitivity or resistance to approved targeted therapies 316 and a tumor type indicator 317 (e.g., soft tissue sarcoma (NOS)) that may all be presented, for example, on the first page of the genomic sequencing clinical report 304B.

Similarly, FIG. 3C illustrates another example of a genomic sequencing clinical report 304C. In certain embodiments, the genomic sequencing clinical report 304C may include a tumor type indicator 322 (e.g., ovary serous carcinoma) and an actionability table 333 on, for example, the first page of the genomic sequencing clinical report 304C. As depicted, the genomic sequencing clinical report 304C may further include, for example, a report highlighting pane 334. In certain embodiments, the report highlighting pane 334 may include, for example, targeted therapies with potential clinical benefit 335 (e.g., targeted therapies associated with National Comprehensive Cancer Network (NCCN) categories of evidence for the ovary serous carcinoma tumor type), genomic findings associated with non-targeted therapies 336 (e.g., gene variants that may inform non-targeted treatment approaches, such as chemotherapy), evidence-matched clinical trial options 338 (e.g., based on the patient's genomic findings and for the ovary serous carcinoma tumor type), and genomic findings with potential germline implications 340.

FIG. 3D illustrates a non-limiting example of one or more pages 300D of the “Genomic Findings” section of a genomic sequencing clinical report with a report highlighting pane. In certain embodiments, the genomic findings with diagnostic implications category 254, the targeted therapies with highest level of evidence category 256, the targeted therapies with potential clinical benefit category 258, the targeted therapies with potential clinical benefit category 260, the targeted therapies with expected resistance category 262, the genomic findings with non-targeted therapy implications category 264, the evidence-matched clinical trial options category 266, the genomic findings with prognostic implications category 268, the genomic findings with germline implications category 270, and the genomic findings with CH implications category 272 may be each associated with one or more embedded links (e.g., uniform resource locator (URL) links or other hyperlinks corresponding to individual page numbers) that may be selected to link a patient, a clinician, or similar user to a landing page (e.g., one or more pages 300E) within the genomic sequencing clinical report that includes a set of detailed information associated with the genomic findings with diagnostic implications category 254, the targeted therapies with highest level of evidence category 256, the targeted therapies with potential clinical benefit category 258, the targeted therapies with potential clinical benefit category 260, the targeted therapies with expected resistance category 262, the genomic findings with non-targeted therapy implications category 264, the evidence-matched clinical trial options category 266, the genomic findings with prognostic implications category 268, the genomic findings with germline implications category 270, and the genomic findings with CH implications category 272.

For example, as depicted, a clinician, a patient, or other similar user may have selected an embedded link corresponding, for example, to the genomic findings with diagnostic implications category 254, the targeted therapies with highest level of evidence category 256, the targeted therapies with potential clinical benefit category 258, the targeted therapies with potential clinical benefit category 260, the targeted therapies with expected resistance category 262, the genomic findings with non-targeted therapy implications category 264, or the genomic findings with CH implications category 272. Thus, the one or more pages 300D may highlight additional detailed data 362 regarding a patient's genomic findings, additional detailed data 364 regarding potential targeted therapy treatment strategies, additional detailed data 366 regarding potential resistance to treatment strategies, additional detailed data 368 regarding non-targeted treatment approaches, additional detail data 370 and 372 regarding potential diagnostic implications, additional detailed data 374 regarding potential CH implications, and so forth.

FIG. 3E illustrates a non-limiting example of an appendix page 300E of a genomic sequencing clinical report with a report highlighting pane. As depicted, the appendix 300E, which may be included on one or more rearward pages of the genomic sequencing clinical report, may include, for example, a high-level overview 376 of all of the information that may be included in the report highlighting pane on the first page of the report. For example, as depicted, the appendix page 300E may reaffirm that that the report highlighting pane is specific to the patient's genomics and tumor type, and may expressly recite that the findings in the report highlighting pane is to be considered in the context of other information in the report and relevant patient information.

FIG. 3F illustrates a non-limiting example of a genomic sequencing clinical report 300F with a report highlighting pane 380. As illustrated, the genomic sequencing clinical report 300F may include an example of a report highlighting pane 380, in which the genomic findings of a patient yields no clinically actionable genomic or medical information. In such an embodiment, the report highlighting pane 380 may present a bullet-point indication 382 that simply notes, for example, that there is no clinically actionable genomic or medical information included in the genomic sequencing clinical report 300F.

In some instances, the gene panel may comprise at least 1, at least 2, at least 3, at least 4, at least 5, at least 6, at least 7, at least 8, at least 9, at least 10, at least 20, at least 30, at least 40, or more than 40 genes.

In some instances, the disclosed methods may be used to generate a report of genomic and medical information associated with a patient by assessing genomic and medical information in at least 1, at least 2, at least 3, at least 4, at least 5, at least 6, at least 7, at least 8, at least 9, at least 10, at least 20, at least 30, at least 40, or more than 40 gene loci.

### METHODS OF USE

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for generating a report of genomic and medical information associated with a patient may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for generating a report of genomic and medical information associated with a patient may be used to select a subject (e.g., a patient) for a clinical trial based on the clinically significant genomic and medical information value determined for one or more gene loci. In some instances, patient selection for clinical trials based on, e.g., identification of clinically significant genomic and medical information at one or more gene loci, may accelerate the development of targeted therapies and improve the healthcare outcomes for treatment decisions.

In some instances, the disclosed methods for generating a report of genomic and medical information associated with a patient may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy (e.g., immunotherapy), surgery, or any combination thereof.

In some instances, the disclosed methods for generating a report of genomic and medical information associated with a patient may be used in treating a disease (e.g., a cancer) in a subject. For example, in response to determining clinically significant genomic and medical information using any of the methods disclosed herein, an effective amount of an anti-cancer therapy or anti-cancer treatment may be administered to the subject.

In some instances, the disclosed methods for generating a report of genomic and medical information associated with a patient may be used for monitoring disease progression or recurrence (e.g., cancer or tumor progression or recurrence) in a subject. For example, in some instances, the methods may be used to determine clinically significant genomic and medical information in a first sample obtained from the subject at a first time point, and used to determine clinically significant genomic and medical information in a second sample obtained from the subject at a second time point, where comparison of the first determination of clinically significant genomic and medical information and the second determination of clinically significant genomic and medical information allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the determination of clinically significant genomic and medical information.

In some instances, the value of clinically significant genomic and medical information determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for generating a report of genomic and medical information associated with a patient may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), a next-generation sequencing (NGS) approach used to assess hundreds of genes (including relevant cancer biomarkers) in a single assay. Inclusion of the disclosed methods for generating a report of genomic and medical information associated with a patient as part of a genomic profiling process (or inclusion of the output from the disclosed methods for generating a report of genomic and medical information associated with a patient as part of the genomic profile of the subject) can improve the validity of, e.g., disease detection calls and treatment decisions, made on the basis of the genomic profile by, for example, independently confirming the presence of clinically significant genomic and medical information in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples include, but are not limited to, a tumor sample, a tissue sample, a biopsy sample, a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavages or bronchoalveolar lavages), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content, e.g., comprising tumor cells or tumor cell nuclei. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g. a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 400, or 500 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or nonspecific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 400, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 500 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 400 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 500 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 400 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D. T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing”, and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLiD, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 400, at least 450, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1470) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized. In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation-detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1e-6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C. A., et al., Genome Res. 2011; 21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix—Bioinformatics. 2010 March 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 500, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 400, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of mutation calling methods is provided in, e.g., International Patent Application Publication No. WO 2020/236941, the entire content of which is incorporated herein by reference.

### Systems

Also disclosed herein are systems designed to implement any of the disclosed methods for generating a report of genomic and medical information associated with a patient in a sample from a subject. The systems may comprise, e.g., one or more processors, and a memory unit communicatively coupled to the one or more processors and configured to store instructions that, when executed by the one or more processors, cause the system perform operations including receiving, at the one or more processors, genomic testing data associated with the patient; based on the genomic testing data, retrieving, at the one or more processors, medical information including one or more potential clinical treatments for the patient; determining, by the one or more processors, that the medical information has at least some clinical significance to the patient; based on at least a portion of the medical information having at least some clinical significance to the patient, generating, by the one or more processors, patient-specific medical data; determining, by the one or more processors, at least one specific position to dispose the medical information on the report based on the patient-specific medical data; and generating, by the one or more processors, the report based on the determined specific position.

In some instances, the disclosed systems may further comprise a sequencer, e.g., a next generation sequencer (also referred to as a massively parallel sequencer). Examples of next generation (or massively parallel) sequencing platforms include, but are not limited to, the Roche 454, Illumina Solexa, ABI-SOLiD, ION Torrent, or Pacific Bioscience sequencing platforms.

In some instances, the disclosed systems may be used for generating a report of genomic and medical information associated with a patient in any of a variety of samples as described herein (e.g., a tissue sample, biopsy sample, hematological sample, or liquid biopsy sample derived from the subject).

In some instances, the plurality of gene loci for which sequencing data is processed to determine clinically significant genomic and medical information may comprise at least 1, 2, 3, 4, 5, 6, 7, 8, 9, 10, or more than 10 gene loci.

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of clinically significant genomic and medical information is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

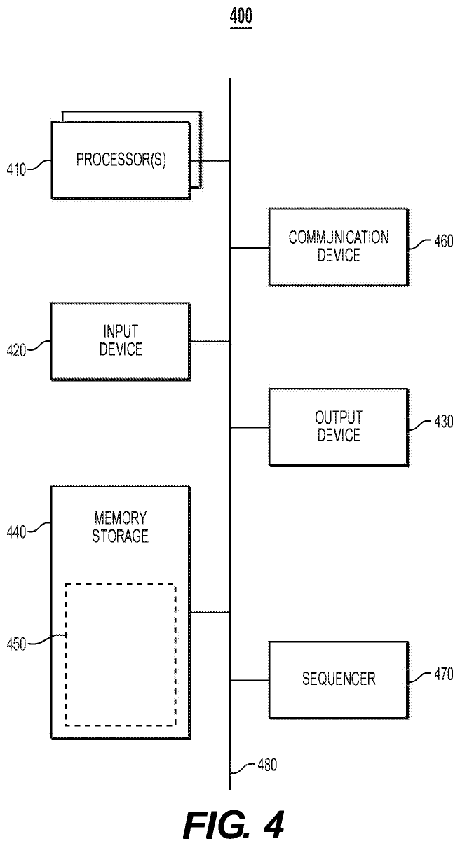

FIG. 4 illustrates an example of a computing device or system in accordance with one embodiment. Device 400 can be a host computer connected to a network. Device 400 can be a client computer or a server. As shown in FIG. 4, device 400 can be any suitable type of microprocessor-based device, such as a personal computer, workstation, server or handheld computing device (portable electronic device) such as a phone or tablet. The device can include, for example, one or more processor(s) 410, input devices 420, output devices 430, memory or storage devices 440, communication devices 460, and nucleic acid sequencers 470. Software 450 residing in memory or storage device 440 may comprise, e.g., an operating system as well as software for executing the methods described herein. Input device 420 and output device 430 can generally correspond to those described herein, and can either be connectable or integrated with the computer.

Input device 420 can be any suitable device that provides input, such as a touch screen, keyboard or keypad, mouse, or voice-recognition device. Output device 430 can be any suitable device that provides output, such as a touch screen, haptics device, or speaker.

Storage 440 can be any suitable device that provides storage (e.g., an electrical, magnetic or optical memory including a RAM (volatile and non-volatile), cache, hard drive, or removable storage disk). Communication device 460 can include any suitable device capable of transmitting and receiving signals over a network, such as a network interface chip or device. The components of the computer can be connected in any suitable manner, such as via a wired media (e.g., a physical system bus 480, Ethernet connection, or any other wire transfer technology) or wirelessly (e.g., Bluetooth®, Wi-Fi®, or any other wireless technology).

Software module 450, which can be stored as executable instructions in storage 450 and executed by processor(s) 410, can include, for example, an operating system and/or the processes that embody the functionality of the methods of the present disclosure (e.g., as embodied in the devices as described herein).

Software module 450 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 440, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 450 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

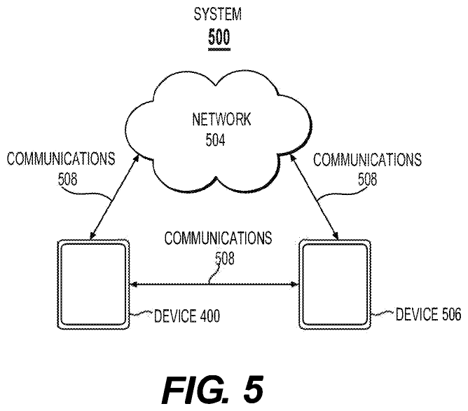

Device 400 may be connected to a network (e.g., network 504, as shown in FIG. 5 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 400 can be implemented using any operating system, e.g., an operating system suitable for operating on the network. Software module 450 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 410.

Device 400 can further include a sequencer 470, which can be any suitable nucleic acid sequencing instrument.

FIG. 5 illustrates an example of a computing system in accordance with one embodiment. In system 500, device 400 (e.g., as described above and illustrated in FIG. 4) is connected to network 504, which is also connected to device 506. In some embodiments, device 506 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system.

Devices 400 and 506 may communicate, e.g., using suitable communication interfaces via network 504, such as a Local Area Network (LAN), Virtual Private Network (VPN), or the Internet. In some embodiments, network 504 can be, for example, the Internet, an intranet, a virtual private network, a cloud network, a wired network, or a wireless network. Devices 400 and 506 may communicate, in part or in whole, via wireless or hardwired communications, such as Ethernet, IEEE 802.11b wireless, or the like. Additionally, devices 400 and 506 may communicate, e.g., using suitable communication interfaces, via a second network, such as a mobile/cellular network. Communication between devices 400 and 506 may further include or communicate with various servers such as a mail server, mobile server, media server, telephone server, and the like. In some embodiments, Devices 400 and 506 can communicate directly (instead of, or in addition to, communicating via network 504), e.g., via wireless or hardwired communications, such as Ethernet, IEEE 802.11b wireless, or the like. In some embodiments, devices 400 and 506 communicate via communications 508, which can be a direct connection or can occur via a network (e.g., network 504).

One or all of devices 400 and 506 generally include logic (e.g., http web server logic) or are programmed to format data, accessed from local or remote databases or other sources of data and content, for providing and/or receiving information via network 504 according to various examples described herein.

### EXAMPLES

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

### Exemplary Implementations

1. A method for generating a report of genomic and medical information associated with a patient, comprising:
   providing a plurality of nucleic acid molecules obtained from a sample from a subject;
   ligating one or more adapters onto one or more nucleic acid molecules from the plurality of nucleic acid molecules;
   amplifying the one or more ligated nucleic acid molecules from the plurality of nucleic acid molecules;
   capturing amplified nucleic acid molecules from the amplified nucleic acid molecules;
   sequencing, by a sequencer, the captured nucleic acid molecules to obtain a plurality of sequence reads that represent the captured nucleic acid molecules;
   receiving, at one or more processors, genomic testing data associated with the patient based on the plurality of sequence reads;
   based on the genomic testing data, retrieving, at the one or more processors, medical information including one or more potential clinical treatments for the patient;
   determining, by the one or more processors, that the medical information has at least some clinical significance to the patient;
   based on at least a portion of the medical information having at least some clinical significance to the patient, generating, by the one or more processors, patient-specific medical data;
   determining, by the one or more processors, at least one specific position to dispose the medical information on the report based on the patient-specific medical data; and
   generating, by the one or more processors, the report based on the determined at least one specific position.
2. The method of Clause 1, wherein receiving the genomic testing data comprises obtaining biomarker testing data, tumor testing data, tumor type, molecular testing data, next-generation sequencing (NGS) data, genomic profiling data, or clinical information.
3. The method of Clause 1, wherein the determined at least one specific position comprises a prominent location on a first page of the report.
4. The method of Clause 1, further comprising:
   comparing the patient genomic testing data to data having diagnostic implications;
   determining whether the patient genomic testing data satisfies a first predetermined criteria based on the data having diagnostic implications, wherein the first predetermined criteria comprises a match of one or more of a gene variant or a biomarker in the medical information;
   based on a determination that the patient genomic testing data and patient tumor type satisfies the first predetermined criteria, generating the patient-specific data to include genomic findings with the diagnostic implications.
5. The method of Clause 4, wherein the medical information includes an annotation indicative of a gene variant and a tumor type, wherein the first predetermined criteria comprises a match of the one or more of the gene variant or the biomarker in the medical information to the annotation.
6. The method of Clause 1, further comprising:
   comparing the patient genomic testing data and a patient tumor type to medical information in a knowledgebase, wherein the medical information in a knowledgebase includes one or more targeted therapies; and
   determining whether the patient genomic testing data and the patient tumor type satisfies a second predetermined criteria based on the medical information in the knowledgebase, wherein the second predetermined criteria comprises a match of the patient tumor type and a gene variant or biomarker in the patient genomic testing data with medical information in the knowledgebase for approved targeted therapies with particular clinical significance;
   based on a determination that the patient genomic testing data and the patient tumor type satisfies the second predetermined criteria, generating patient-specific medical data of clinically significant targeted therapies with potential clinical benefit;
   if the patient genomic testing data and the patient tumor type satisfies the second predetermined criteria for more than one of the clinically significant targeted therapies, reducing the patient-specific medical data to include only approved therapies with the highest level of evidence.
7. The method of Clause 6, wherein the medical information in the knowledgebase for approved targeted therapies with particular clinical significance includes an annotation, and wherein the second predetermined criteria comprises a match of the patient tumor type and the gene variant or biomarker to the annotation.
8. The method of Clause 1, further comprising:
   comparing the patient genomic testing data and a patient tumor type to medical information in a knowledgebase for targeted therapies with expected resistance;
   determining whether the patient genomic testing data and the patient tumor type satisfies a third predetermined criteria based on the medical information in the knowledgebase, wherein the third predetermined criteria comprises a match of the patient tumor type and a gene variant or biomarker in the patient genomic testing data with medical information in the knowledgebase for targeted therapies with expected resistance with particular clinical significance;
   based on a determination that the patient genomic testing data and the patient tumor type satisfies the third predetermined criteria, generating patient-specific medical data of clinically significant targeted therapies with expected resistance.
9. The method of Clause 8, wherein the medical information in the knowledgebase for targeted therapies with expected resistance with particular clinical significance include an annotation, and wherein the third predetermined criteria comprises a match of the patient tumor type and the gene variant or biomarker to the annotation.
10. The method of Clause 1, further comprising:
   comparing the patient genomic testing data and a patient tumor type to data in a knowledgebase for genomic findings with non-targeted therapy implications;
   determining whether the patient genomic testing data and the patient tumor type satisfies a fourth predetermined criteria based on the data in the knowledgebase, wherein the fourth predetermined criteria comprises a match of the patient tumor type and a gene variant or biomarker in the patient genomic testing data with data in the knowledgebase for genomic findings with non-targeted therapy implications with particular clinical significance;
   based on a determination that the patient genomic testing data and the patient tumor type satisfies the fourth predetermined criteria, generating patient-specific medical data of clinically significant genomic findings with non-targeted therapy implications.
11. The method of Clause 10, wherein the data in the knowledgebase for genomic findings with non-targeted therapy implications with particular clinical significance, and wherein the fourth predetermined criteria comprises a match of the patient tumor type and the gene variant or biomarker to the annotation.
12. The method of Clause 1, further comprising:
   comparing the patient genomic testing data, a patient tumor type, and a patient age to clinical trial data in a knowledgebase;
   determining whether the patient genomic testing data, the patient tumor type, and the patient age satisfies a fifth predetermined criteria based on the clinical trial data in the knowledgebase, wherein the fifth predetermined criteria comprises a match of the patient tumor type, the patient age, and a gene variant or biomarker in the patient genomic testing data with the clinical trial data in the knowledgebase;
   based on a determination that the patient genomic testing data, the patient tumor type, and the patient age satisfies the fifth predetermined criteria, generating patient-specific medical data of clinically significant evidence-matched clinical trials.
13. The method of Clause 1, further comprising:
   comparing the patient genomic testing data and a patient tumor type to data in a knowledgebase for genomic findings with prognostic implications;
   determining whether the patient genomic testing data and the patient tumor type satisfies a sixth predetermined criteria based on the data in the knowledgebase, wherein the sixth predetermined criteria comprises a match of the patient tumor type and a gene variant or biomarker in the patient genomic testing data with data in the knowledgebase for genomic finding with prognostic implications with particular clinical significance;
   based on a determination that the patient genomic testing data and the patient tumor type satisfies the sixth predetermined criteria, generating patient-specific medical data of clinically significant genomic findings with prognostic implications.
14. The method of Clause 13, wherein the data in the knowledgebase for genomic finding with prognostic implications with particular clinical significance include an annotation, and wherein the sixth predetermined criteria comprises a match of the patient tumor type and the gene variant or biomarker to the annotation.
15. The method of Clause 1, further comprising:
   comparing the patient genomic testing data and a patient tumor type to data in a knowledgebase for genomic findings with potential germline implications;
   determining whether the patient genomic testing data and the patient tumor type satisfies a seventh predetermined criteria based on the data in the knowledgebase, wherein the seventh predetermined criteria comprises a 1) a match of a potential germline mutation associated with one or more hereditary cancer predisposition syndromes associated with germline implications in the patient genomic testing data, and 2) a determination that the potential germline mutation is above a predetermined variant allele frequency (VAF) threshold in the patient genomic testing data;
   based on a determination that the patient genomic testing data and the patient tumor type satisfies the seventh predetermined criteria, generating patient-specific medical data of clinically significant genomic findings with potential germline implications to be considered for additional germline testing in a clinical context.
16. The method of Clause 15, wherein the data in the knowledgebase for genomic findings with potential germline implications include an annotation, and wherein the seventh predetermined criteria comprises a match of at least the potential germline mutation associated with one or more hereditary cancer predisposition syndromes associated with germline implications to the annotation.
17. The method of Clause 1, further comprising:
   comparing the patient genomic testing data and a patient tumor type to data in a knowledgebase for genomic findings with potential clonal hematopoiesis (CH) implications;
   determining whether the patient genomic testing data and the patient tumor type satisfies an eighth predetermined criteria based on the data in the knowledgebase, wherein the eighth predetermined criteria comprises: 1) a match of a genomic finding with potential CH implications when identified in the patient tumor type, and 2) a determination that the matched genomic finding with potential CH implications is identified in patient genomic testing data from one or more particular analytes;
   based on a determination that the patient genomic testing data and the patient tumor type satisfies the eighth predetermined criteria, generating the patient-specific highlight of clinically significant genomic findings with potential CH implications.
18. A method for generating a report of genomic and medical information associated with a patient, comprising:
   receiving, at one or more processors, genomic testing data associated with the patient;
   based on the genomic testing data, retrieving, at the one or more processors, medical information including one or more potential clinical treatments for the patient;
   determining, by the one or more processors, that the medical information has at least some clinical significance to the patient;
   based on at least a portion of the medical information having at least some clinical significance to the patient, generating, by the one or more processors, patient-specific medical data;
   determining, by the one or more processors, at least one specific position to dispose the medical information on the report based on the patient-specific medical data; and
   generating, by the one or more processors, the report based on the determined at least one specific position.
19. The method of Clause 18, wherein receiving the genomic testing data comprises obtaining biomarker testing data, tumor testing data, tumor type, molecular testing data, next-generation sequencing (NGS) data, genomic profiling data, or clinical information.
20. The method of Clause 18, wherein retrieving medical information including one or more potential clinical treatments comprises retrieving information from one or more of scientific literature or medical literature, the method further comprising:
   inputting one or more of the retrieved scientific literature or the retrieved medical literature to a trained machine-learning model to obtain a prediction of the patient-specific medical data based on the set of scientific and medical literature.
21. The method of Clause 18, wherein a predetermined set of genomic or therapeutic categories comprises a genomic findings with diagnostic implications category, a targeted therapies with highest level of evidence category, a targeted therapies with expected resistance category, a genomic findings with non-targeted therapy implications category, an evidence-matched clinical trial options category, a genomic findings with prognostic implications category, a genomic findings with potential germline implications category, a genomic findings with potential clonal hematopoiesis (CH) implications category, a genomic findings with companion diagnostic (CDx) category, a targeted therapies with pan-tumor indications category, or an investigational targeted therapies category.
22. The method of Clause 18, wherein the medical information includes relative clinical significances based on genomic information, and wherein the patient-specific medical data is ranked according to the relative clinical significances based on the patient genomic testing data.
23. The method of Clause 18, further comprising:
   comparing the patient genomic testing data to data having diagnostic implications;
   determining whether the patient genomic testing data satisfies a first predetermined criteria based on the data having diagnostic implications, wherein the first predetermined criteria comprises a match of one or more of a gene variant or a biomarker in the medical information;
   based on a determination that the patient genomic testing data and patient tumor type satisfies the first predetermined criteria, generating the patient-specific data to include genomic findings with the diagnostic implications.
24. The method of Clause 23, wherein the medical information includes an annotation indicative of a gene variant and a tumor type, wherein the first predetermined criteria comprises a match of the one or more of the gene variant or the biomarker in the medical information to the annotation.
25. The method of Clause 18, further comprising:
   comparing the patient genomic testing data and a patient tumor type to medical information in a knowledgebase, wherein the medical information in a knowledgebase includes one or more targeted therapies; and
   determining whether the patient genomic testing data and the patient tumor type satisfies a second predetermined criteria based on the medical information in the knowledgebase, wherein the second predetermined criteria comprises a match of the patient tumor type and a gene variant or biomarker in the patient genomic testing data with medical information in the knowledgebase for approved targeted therapies with particular clinical significance;
   based on a determination that the patient genomic testing data and the patient tumor type satisfies the second predetermined criteria, generating patient-specific medical data of clinically significant targeted therapies with potential clinical benefit;
   if the patient genomic testing data and the patient tumor type satisfies the second predetermined criteria for more than one of the clinically significant targeted therapies, reducing the patient-specific medical data to include only approved therapies with the highest level of evidence.
26. The method of Clause 25, wherein the medical information in the knowledgebase for approved targeted therapies with particular clinical significance includes an annotation, and wherein the second predetermined criteria comprises a match of the patient tumor type and the gene variant or biomarker to the annotation.
27. The method of Clause 18, further comprising:
   comparing the patient genomic testing data and a patient tumor type to medical information in a knowledgebase for targeted therapies with expected resistance;
   determining whether the patient genomic testing data and the patient tumor type satisfies a third predetermined criteria based on the medical information in the knowledgebase, wherein the third predetermined criteria comprises a match of the patient tumor type and a gene variant or biomarker in the patient genomic testing data with medical information in the knowledgebase for targeted therapies with expected resistance with particular clinical significance;
   based on a determination that the patient genomic testing data and the patient tumor type satisfies the third predetermined criteria, generating patient-specific medical data of clinically significant targeted therapies with expected resistance.
28. The method of Clause 27, wherein the medical information in the knowledgebase for targeted therapies with expected resistance with particular clinical significance include an annotation, and wherein the third predetermined criteria comprises a match of the patient tumor type and the gene variant or biomarker to the annotation.
29. The method of Clause 18, further comprising:
   comparing the patient genomic testing data and a patient tumor type to data in a knowledgebase for genomic findings with non-targeted therapy implications;
   determining whether the patient genomic testing data and the patient tumor type satisfies a fourth predetermined criteria based on the data in the knowledgebase, wherein the fourth predetermined criteria comprises a match of the patient tumor type and a gene variant or biomarker in the patient genomic testing data with data in the knowledgebase for genomic findings with non-targeted therapy implications with particular clinical significance;
   based on a determination that the patient genomic testing data and the patient tumor type satisfies the fourth predetermined criteria, generating patient-specific medical data of clinically significant genomic findings with non-targeted therapy implications.
30. The method of Clause 29, wherein the data in the knowledgebase for genomic findings with non-targeted therapy implications with particular clinical significance, and wherein the fourth predetermined criteria comprises a match of the patient tumor type and the gene variant or biomarker to the annotation.
31. The method of Clause 18, further comprising:
   comparing the patient genomic testing data, a patient tumor type, and a patient age to clinical trial data in a knowledgebase;
   determining whether the patient genomic testing data, the patient tumor type, and the patient age satisfies a fifth predetermined criteria based on the clinical trial data in the knowledgebase, wherein the fifth predetermined criteria comprises a match of the patient tumor type, the patient age, and a gene variant or biomarker in the patient genomic testing data with the clinical trial data in the knowledgebase;
   based on a determination that the patient genomic testing data, the patient tumor type, and the patient age satisfies the fifth predetermined criteria, generating patient-specific medical data of clinically significant evidence-matched clinical trials.
32. The method of Clause 31, wherein the fifth predetermined criteria is indicative of a gene variant or biomarker, a tumor type, a patient age, and a clinical trial patient recruitment criteria.
33. The method of Clause 18, further comprising:
   comparing the patient genomic testing data and a patient tumor type to data in a knowledgebase for genomic findings with prognostic implications;
   determining whether the patient genomic testing data and the patient tumor type satisfies a sixth predetermined criteria based on the data in the knowledgebase, wherein the sixth predetermined criteria comprises a match of the patient tumor type and a gene variant or biomarker in the patient genomic testing data with data in the knowledgebase for genomic finding with prognostic implications with particular clinical significance;
   based on a determination that the patient genomic testing data and the patient tumor type satisfies the sixth predetermined criteria, generating patient-specific medical data of clinically significant genomic findings with prognostic implications.
34. The method of Clause 33, wherein the data in the knowledgebase for genomic finding with prognostic implications with particular clinical significance include an annotation, and wherein the sixth predetermined criteria comprises a match of the patient tumor type and the gene variant or biomarker to the annotation.
35. The method of Clause 18, further comprising:
   comparing the patient genomic testing data and a patient tumor type to data in a knowledgebase for genomic findings with potential germline implications;
   determining whether the patient genomic testing data and the patient tumor type satisfies a seventh predetermined criteria based on the data in the knowledgebase, wherein the seventh predetermined criteria comprises a 1) a match of a potential germline mutation associated with one or more hereditary cancer predisposition syndromes associated with germline implications in the patient genomic testing data, and 2) a determination that the potential germline mutation is above a predetermined variant allele frequency (VAF) threshold in the patient genomic testing data;
   based on a determination that the patient genomic testing data and the patient tumor type satisfies the seventh predetermined criteria, generating patient-specific medical data of clinically significant genomic findings with potential germline implications to be considered for additional germline testing in a clinical context.
36. The method of Clause 35, wherein the data in the knowledgebase for genomic findings with potential germline implications include an annotation, and wherein the seventh predetermined criteria comprises a match of at least the potential germline mutation associated with one or more hereditary cancer predisposition syndromes associated with germline implications to the annotation.
37. The method of Clause 18, wherein generating the report comprises generating a companion diagnostic (CDx) report associated with the patient.
38. The method of Clause 18, further comprising causing an electronic device to display the report.
39. The method of Clause 38, wherein causing the electronic device to display the report comprises causing a human machine interface (HMI) associated with a clinician to display the report.
40. The method of Clause 38, wherein causing the electronic device to display the report comprises causing an electronic device associated with the patient to display the report.
41. The method of Clause 38, further comprising causing the electronic device to display the report including a predefined area for one or more natural-language phrases on a first page of the report.
42. The method of Clause 41, wherein the one or more natural-language phrases comprises an embedded hyperlink associated with the one or more natural-language phrases.
43. The method of Clause 42, wherein the embedded hyperlink comprises a link to one or more other pages of the report, the one or more other pages including a set of detailed information corresponding to the one or more natural-language phrases.
44. The method of Clause 43, wherein the embedded hyperlink comprises a page number of the one or more other pages.
45. The method of Clause 18, further comprising causing, by the one or more processors, an electronic device to display the report including one or more natural-language phrases in a second predefined area.
46. The method of Clause 45, wherein the second predefined area comprises an appendix on one or more rearward pages of the report.
47. The method of Clause 46, wherein the appendix comprises the one or more natural-language phrases displayed along with additional contextual information determined to be relevant to the patient.
48. A system, comprising:
   one or more non-transitory computer-readable storage media including instructions; and
   one or more processors coupled to the one or more storage media, the one or more processors configured to execute the instructions for:
      receiving, at the one or more processors, genomic testing data associated with the patient;
      based on the genomic testing data, retrieving, at the one or more processors, medical information including one or more potential clinical treatments for the patient;
      determining, by the one or more processors, that the medical information has at least some clinical significance to the patient;
      based on at least a portion of the medical information having at least some clinical significance to the patient, generating, by the one or more processors, patient-specific medical data;
      determining, by the one or more processors, at least one specific position to dispose the medical information on the report based on the patient-specific medical data; and
      generating, by the one or more processors, the report based on the determined at least one specific position.
49. The system of Clause 48, wherein receiving the genomic testing data comprises obtaining biomarker testing data, tumor testing data, tumor type, molecular testing data, next-generation sequencing (NGS) data, genomic profiling data, or clinical information.
50. The system of Claim 48, wherein retrieving medical information including one or more potential clinical treatments comprises retrieving information from one or more of scientific literature or medical literature, the method further comprising:
   inputting one or more of the retrieved scientific literature or the retrieved medical literature to a trained machine-learning model to obtain a prediction of the patient-specific medical data based on the set of scientific and medical literature.
51. The system of Clause 48, wherein a predetermined set of genomic or therapeutic categories comprises a genomic findings with diagnostic implications category, a targeted therapies with highest level of evidence category, a targeted therapies with expected resistance category, a genomic findings with non-targeted therapy implications category, an evidence-matched clinical trial options category, a genomic findings with prognostic implications category, a genomic findings with potential germline implications category, a genomic findings with potential clonal hematopoiesis (CH) implications category, a genomic findings with companion diagnostic (CDx) category, a targeted therapies with pan-tumor indications category, or an investigational targeted therapies category.
52. The system of Clause 48, wherein the medical information includes relative clinical significances based on genomic information, and wherein the patient-specific medical data is ranked according to the relative clinical significances based on the patient genomic testing data.
53. The system of Clause 48, further comprising:
   comparing the patient genomic testing data to data having diagnostic implications;
   determining whether the patient genomic testing data satisfies a first predetermined criteria based on the data having diagnostic implications, wherein the first predetermined criteria comprises a match of one or more of a gene variant or a biomarker in the medical information;
   based on a determination that the patient genomic testing data and patient tumor type satisfies the first predetermined criteria, generating the patient-specific data to include genomic findings with the diagnostic implications.
54. The system of Clause 53, wherein the medical information includes an annotation indicative of a gene variant and a tumor type, wherein the first predetermined criteria comprises a match of the one or more of the gene variant or the biomarker in the medical information to the annotation.
55. The system of Clause 48, further comprising:
   comparing the patient genomic testing data and a patient tumor type to medical information in a knowledgebase, wherein the medical information in a knowledgebase includes one or more targeted therapies; and
   determining whether the patient genomic testing data and the patient tumor type satisfies a second predetermined criteria based on the medical information in the knowledgebase, wherein the second predetermined criteria comprises a match of the patient tumor type and a gene variant or biomarker in the patient genomic testing data with medical information in the knowledgebase for approved targeted therapies with particular clinical significance;
   based on a determination that the patient genomic testing data and the patient tumor type satisfies the second predetermined criteria, generating patient-specific medical data of clinically significant targeted therapies with potential clinical benefit;
   if the patient genomic testing data and the patient tumor type satisfies the second predetermined criteria for more than one of the clinically significant targeted therapies, reducing the patient-specific medical data to include only approved therapies with the highest level of evidence.
56. The system of Clause 55, wherein the medical information in the knowledgebase for approved targeted therapies with particular clinical significance includes an annotation, and wherein the second predetermined criteria comprises a match of the patient tumor type and the gene variant or biomarker to the annotation.
57. The system of Clause 48, further comprising:
   comparing the patient genomic testing data and a patient tumor type to medical information in a knowledgebase for targeted therapies with expected resistance;
   determining whether the patient genomic testing data and the patient tumor type satisfies a third predetermined criteria based on the medical information in the knowledgebase, wherein the third predetermined criteria comprises a match of the patient tumor type and a gene variant or biomarker in the patient genomic testing data with medical information in the knowledgebase for targeted therapies with expected resistance with particular clinical significance;
   based on a determination that the patient genomic testing data and the patient tumor type satisfies the third predetermined criteria, generating patient-specific medical data of clinically significant targeted therapies with expected resistance.
58. The system of Clause 57, wherein the medical information in the knowledgebase for targeted therapies with expected resistance with particular clinical significance include an annotation, and wherein the third predetermined criteria comprises a match of the patient tumor type and the gene variant or biomarker to the annotation.
59. The system of Clause 48, further comprising:
   comparing the patient genomic testing data and a patient tumor type to data in a knowledgebase for genomic findings with non-targeted therapy implications;
   determining whether the patient genomic testing data and the patient tumor type satisfies a fourth predetermined criteria based on the data in the knowledgebase, wherein the fourth predetermined criteria comprises a match of the patient tumor type and a gene variant or biomarker in the patient genomic testing data with data in the knowledgebase for genomic findings with non-targeted therapy implications with particular clinical significance;
   based on a determination that the patient genomic testing data and the patient tumor type satisfies the fourth predetermined criteria, generating patient-specific medical data of clinically significant genomic findings with non-targeted therapy implications.
60. The system of Clause 59, wherein the data in the knowledgebase for genomic findings with non-targeted therapy implications with particular clinical significance, and wherein the fourth predetermined criteria comprises a match of the patient tumor type and the gene variant or biomarker to the annotation.
61. The system of Clause 48, further comprising:
   comparing the patient genomic testing data, a patient tumor type, and a patient age to clinical trial data in a knowledgebase;
   determining whether the patient genomic testing data, the patient tumor type, and the patient age satisfies a fifth predetermined criteria based on the clinical trial data in the knowledgebase, wherein the fifth predetermined criteria comprises a match of the patient tumor type, the patient age, and a gene variant or biomarker in the patient genomic testing data with the clinical trial data in the knowledgebase;
   based on a determination that the patient genomic testing data, the patient tumor type, and the patient age satisfies the fifth predetermined criteria, generating patient-specific medical data of clinically significant evidence-matched clinical trials.
62. The system of Clause 61, wherein the fifth predetermined criteria is indicative of a gene variant or biomarker, a tumor type, a patient age, and a clinical trial patient recruitment criteria.
63. The system of Clause 48, further comprising:
   comparing the patient genomic testing data and a patient tumor type to data in a knowledgebase for genomic findings with prognostic implications;
   determining whether the patient genomic testing data and the patient tumor type satisfies a sixth predetermined criteria based on the data in the knowledgebase, wherein the sixth predetermined criteria comprises a match of the patient tumor type and a gene variant or biomarker in the patient genomic testing data with data in the knowledgebase for genomic finding with prognostic implications with particular clinical significance;
   based on a determination that the patient genomic testing data and the patient tumor type satisfies the sixth predetermined criteria, generating patient-specific medical data of clinically significant genomic findings with prognostic implications.
64. The system of Clause 63, wherein the data in the knowledgebase for genomic finding with prognostic implications with particular clinical significance include an annotation, and wherein the sixth predetermined criteria comprises a match of the patient tumor type and the gene variant or biomarker to the annotation.
65. The system of Clause 48, further comprising:
   comparing the patient genomic testing data and a patient tumor type to data in a knowledgebase for genomic findings with potential germline implications;
   determining whether the patient genomic testing data and the patient tumor type satisfies a seventh predetermined criteria based on the data in the knowledgebase, wherein the seventh predetermined criteria comprises a 1) a match of a potential germline mutation associated with one or more hereditary cancer predisposition syndromes associated with germline implications in the patient genomic testing data, and 2) a determination that the potential germline mutation is above a predetermined variant allele frequency (VAF) threshold in the patient genomic testing data;
   based on a determination that the patient genomic testing data and the patient tumor type satisfies the seventh predetermined criteria, generating patient-specific medical data of clinically significant genomic findings with potential germline implications to be considered for additional germline testing in a clinical context.
66. The system of Clause 65, wherein the data in the knowledgebase for genomic findings with potential germline implications include an annotation, and wherein the seventh predetermined criteria comprises a match of at least the potential germline mutation associated with one or more hereditary cancer predisposition syndromes associated with germline implications to the annotation.
67. The system of Clause 48, wherein generating the report comprises generating a companion diagnostic (CDx) report associated with the patient.
68. The system of Clause 48, further comprising causing an electronic device to display the report.
69. The system of Clause 68, wherein causing the electronic device to display the report comprises causing a human machine interface (HMI) associated with a clinician to display the report.
70. The system of Clause 68, wherein causing the electronic device to display the report comprises causing an electronic device associated with the patient to display the report.
71. The system of Clause 68, further comprising causing the electronic device to display the report including a predefined area for one or more natural-language phrases on a first page of the report.
72. The system of Clause 71, wherein the one or more natural-language phrases comprises an embedded hyperlink associated with the one or more natural-language phrases.
73. The system of Clause 72, wherein the embedded hyperlink comprises a link to one or more other pages of the report, the one or more other pages including a set of detailed information corresponding to the one or more natural-language phrases.
74. The system of Clause 73, wherein the embedded hyperlink comprises a page number of the one or more other pages.
75. The system of Clause 48, further comprising causing, by the one or more processors, an electronic device to display the report including one or more natural-language phrases in a second predefined area.
76. The system of Clause 75, wherein the second predefined area comprises an appendix on one or more rearward pages of the report.
77. The system of Clause 76, wherein the appendix comprises the one or more natural-language phrases displayed along with additional contextual information determined to be relevant to the patient.
78. A non-transitory computer-readable medium comprising instructions that, when executed by one or more processors, cause the one or more processors to perform operations including:
   receiving, at the one or more processors, genomic testing data associated with the patient;
   based on the genomic testing data, retrieving, at the one or more processors, medical information including one or more potential clinical treatments for the patient;
   determining, by the one or more processors, that the medical information has at least some clinical significance to the patient;
   based on at least a portion of the medical information having at least some clinical significance to the patient, generating, by the one or more processors, patient-specific medical data;
   determining, by the one or more processors, at least one specific position to dispose the medical information on the report based on the patient-specific medical data; and
   generating, by the one or more processors, the report based on the determined at least one specific position.
79. The non-transitory computer-readable medium of Clause 78, wherein receiving the genomic testing data comprises obtaining biomarker testing data, tumor testing data, tumor type, molecular testing data, next-generation sequencing (NGS) data, genomic profiling data, or clinical information.
80. The non-transitory computer-readable medium of Clause 78, wherein retrieving medical information including one or more potential clinical treatments comprises retrieving information from one or more of scientific literature or medical literature, the method further comprising:
   inputting one or more of the retrieved scientific literature or the retrieved medical literature to a trained machine-learning model to obtain a prediction of the patient-specific medical data based on the set of scientific and medical literature.
81. The non-transitory computer-readable medium of Clause 78, wherein a predetermined set of genomic or therapeutic categories comprises a genomic findings with diagnostic implications category, a targeted therapies with highest level of evidence category, a targeted therapies with expected resistance category, a genomic findings with non-targeted therapy implications category, an evidence-matched clinical trial options category, a genomic findings with prognostic implications category, a genomic findings with potential germline implications category, a genomic findings with potential clonal hematopoiesis (CH) implications category, a genomic findings with companion diagnostic (CDx) category, a targeted therapies with pan-tumor indications category, or an investigational targeted therapies category.
82. The non-transitory computer-readable medium of Clause 78, wherein the medical information includes relative clinical significances based on genomic information, and wherein the patient-specific medical data is ranked according to the relative clinical significances based on the patient genomic testing data.
83. The non-transitory computer-readable medium of Clause 78, further comprising:
   comparing the patient genomic testing data to data having diagnostic implications;
   determining whether the patient genomic testing data satisfies a first predetermined criteria based on the data having diagnostic implications, wherein the first predetermined criteria comprises a match of one or more of a gene variant or a biomarker in the medical information;
   based on a determination that the patient genomic testing data and patient tumor type satisfies the first predetermined criteria, generating the patient-specific data to include genomic findings with the diagnostic implications.
84. The non-transitory computer-readable medium of Clause 83, wherein the medical information includes an annotation indicative of a gene variant and a tumor type, wherein the first predetermined criteria comprises a match of the one or more of the gene variant or the biomarker in the medical information to the annotation.
85. The non-transitory computer-readable medium of Clause 78, further comprising:
   comparing the patient genomic testing data and a patient tumor type to medical information in a knowledgebase, wherein the medical information in a knowledgebase includes one or more targeted therapies; and
   determining whether the patient genomic testing data and the patient tumor type satisfies a second predetermined criteria based on the medical information in the knowledgebase, wherein the second predetermined criteria comprises a match of the patient tumor type and a gene variant or biomarker in the patient genomic testing data with medical information in the knowledgebase for approved targeted therapies with particular clinical significance;
   based on a determination that the patient genomic testing data and the patient tumor type satisfies the second predetermined criteria, generating patient-specific medical data of clinically significant targeted therapies with potential clinical benefit;
   if the patient genomic testing data and the patient tumor type satisfies the second predetermined criteria for more than one of the clinically significant targeted therapies, reducing the patient-specific medical data to include only approved therapies with the highest level of evidence.
86. The non-transitory computer-readable medium of Clause 85, wherein the medical information in the knowledgebase for approved targeted therapies with particular clinical significance includes an annotation, and wherein the second predetermined criteria comprises a match of the patient tumor type and the gene variant or biomarker to the annotation.
87. The non-transitory computer-readable medium of Clause 78, further comprising:
   comparing the patient genomic testing data and a patient tumor type to medical information in a knowledgebase for targeted therapies with expected resistance;
   determining whether the patient genomic testing data and the patient tumor type satisfies a third predetermined criteria based on the medical information in the knowledgebase, wherein the third predetermined criteria comprises a match of the patient tumor type and a gene variant or biomarker in the patient genomic testing data with medical information in the knowledgebase for targeted therapies with expected resistance with particular clinical significance;
   based on a determination that the patient genomic testing data and the patient tumor type satisfies the third predetermined criteria, generating patient-specific medical data of clinically significant targeted therapies with expected resistance.
88. The non-transitory computer-readable medium of Clause 87, wherein the medical information in the knowledgebase for targeted therapies with expected resistance with particular clinical significance include an annotation, and wherein the third predetermined criteria comprises a match of the patient tumor type and the gene variant or biomarker to the annotation.
89. The non-transitory computer-readable medium of Clause 78, further comprising:
   comparing the patient genomic testing data and a patient tumor type to data in a knowledgebase for genomic findings with non-targeted therapy implications;
   determining whether the patient genomic testing data and the patient tumor type satisfies a fourth predetermined criteria based on the data in the knowledgebase, wherein the fourth predetermined criteria comprises a match of the patient tumor type and a gene variant or biomarker in the patient genomic testing data with data in the knowledgebase for genomic findings with non-targeted therapy implications with particular clinical significance;
   based on a determination that the patient genomic testing data and the patient tumor type satisfies the fourth predetermined criteria, generating patient-specific medical data of clinically significant genomic findings with non-targeted therapy implications.
90. The non-transitory computer-readable medium of Clause 89, wherein the data in the knowledgebase for genomic findings with non-targeted therapy implications with particular clinical significance, and wherein the fourth predetermined criteria comprises a match of the patient tumor type and the gene variant or biomarker to the annotation.
91. The non-transitory computer-readable medium of Claim 78, further comprising:
   comparing the patient genomic testing data, a patient tumor type, and a patient age to clinical trial data in a knowledgebase;
   determining whether the patient genomic testing data, the patient tumor type, and the patient age satisfies a fifth predetermined criteria based on the clinical trial data in the knowledgebase, wherein the fifth predetermined criteria comprises a match of the patient tumor type, the patient age, and a gene variant or biomarker in the patient genomic testing data with the clinical trial data in the knowledgebase;
   based on a determination that the patient genomic testing data, the patient tumor type, and the patient age satisfies the fifth predetermined criteria, generating patient-specific medical data of clinically significant evidence-matched clinical trials.
92. The non-transitory computer-readable medium of Clause 91, wherein the fifth predetermined criteria is indicative of a gene variant or biomarker, a tumor type, a patient age, and a clinical trial patient recruitment criteria.
93. The non-transitory computer-readable medium of Clause 78, further comprising:
   comparing the patient genomic testing data and a patient tumor type to data in a knowledgebase for genomic findings with prognostic implications;
   determining whether the patient genomic testing data and the patient tumor type satisfies a sixth predetermined criteria based on the data in the knowledgebase, wherein the sixth predetermined criteria comprises a match of the patient tumor type and a gene variant or biomarker in the patient genomic testing data with data in the knowledgebase for genomic finding with prognostic implications with particular clinical significance;
   based on a determination that the patient genomic testing data and the patient tumor type satisfies the sixth predetermined criteria, generating patient-specific medical data of clinically significant genomic findings with prognostic implications.
94. The non-transitory computer-readable medium of Clause 93, wherein the data in the knowledgebase for genomic finding with prognostic implications with particular clinical significance include an annotation, and wherein the sixth predetermined criteria comprises a match of the patient tumor type and the gene variant or biomarker to the annotation.
95. The non-transitory computer-readable medium of Clause 78, further comprising:
   comparing the patient genomic testing data and a patient tumor type to data in a knowledgebase for genomic findings with potential germline implications;
   determining whether the patient genomic testing data and the patient tumor type satisfies a seventh predetermined criteria based on the data in the knowledgebase, wherein the seventh predetermined criteria comprises a 1) a match of a potential germline mutation associated with one or more hereditary cancer predisposition syndromes associated with germline implications in the patient genomic testing data, and 2) a determination that the potential germline mutation is above a predetermined variant allele frequency (VAF) threshold in the patient genomic testing data;
   based on a determination that the patient genomic testing data and the patient tumor type satisfies the seventh predetermined criteria, generating patient-specific medical data of clinically significant genomic findings with potential germline implications to be considered for additional germline testing in a clinical context.
96. The non-transitory computer-readable medium of Clause 95, wherein the data in the knowledgebase for genomic findings with potential germline implications include an annotation, and wherein the seventh predetermined criteria comprises a match of at least the potential germline mutation associated with one or more hereditary cancer predisposition syndromes associated with germline implications to the annotation.
97. The non-transitory computer-readable medium of Clause 78, wherein generating the report comprises generating a companion diagnostic (CDx) report associated with the patient.
98. The non-transitory computer-readable medium of Clause 78, further comprising causing an electronic device to display the report.
99. The non-transitory computer-readable medium of Clause 98, wherein causing the electronic device to display the report comprises causing a human machine interface (HMI) associated with a clinician to display the report.
100. The non-transitory computer-readable medium of Clause 98, wherein causing the electronic device to display the report comprises causing an electronic device associated with the patient to display the report.
101. The non-transitory computer-readable medium of Clause 98, further comprising causing the electronic device to display the report including a predefined area for one or more natural-language phrases on a first page of the report.
102. The non-transitory computer-readable medium of Clause 101, wherein the one or more natural-language phrases comprises an embedded hyperlink associated with the one or more natural-language phrases.
103. The non-transitory computer-readable medium of Clause 102, wherein the embedded hyperlink comprises a link to one or more other pages of the report, the one or more other pages including a set of detailed information corresponding to the one or more natural-language phrases.
104. The non-transitory computer-readable medium of Clause 103, wherein the embedded hyperlink comprises a page number of the one or more other pages.
105. The non-transitory computer-readable medium of Clause 78, further comprising causing, by the one or more processors, an electronic device to display the report including one or more natural-language phrases in a second predefined area.
106. The non-transitory computer-readable medium of Clause 105, wherein the second predefined area comprises an appendix on one or more rearward pages of the report.
107. The non-transitory computer-readable medium of Clause 106, wherein the appendix comprises the one or more natural-language phrases displayed along with additional contextual information determined to be relevant to the patient.

1. A method for generating a report of genomic and medical information associated with a patient, comprising:
receiving, at one or more processors, genomic testing data associated with the patient;
based on the genomic testing data, retrieving, at the one or more processors, medical information including one or more potential clinical treatments for the patient;
determining, by the one or more processors, that the medical information has at least some clinical significance to the patient;
based on at least a portion of the medical information having at least some clinical significance to the patient, generating, by the one or more processors, patient-specific medical data;
determining, by the one or more processors, at least one specific position to dispose the medical information on the report based on the patient-specific medical data; and
generating, by the one or more processors, the report based on the determined at least one specific position.

2. The method of claim 1, wherein receiving the genomic testing data comprises obtaining biomarker testing data, tumor testing data, tumor type, molecular testing data, next-generation sequencing (NGS) data, genomic profiling data, or clinical information.

3. The method of claim 1, wherein retrieving medical information including one or more potential clinical treatments comprises retrieving information from one or more of scientific literature or medical literature, the method further comprising:
inputting one or more of the retrieved scientific literature or the retrieved medical literature to a trained machine-learning model to obtain a prediction of the patient-specific medical data based on the set of scientific and medical literature.

4. The method of claim 1, wherein a predetermined set of genomic or therapeutic categories comprises a genomic findings with diagnostic implications category, a targeted therapies with highest level of evidence category, a targeted therapies with expected resistance category, a genomic findings with non-targeted therapy implications category, an evidence-matched clinical trial options category, a genomic findings with prognostic implications category, a genomic findings with potential germline implications category, a genomic findings with potential clonal hematopoiesis (CH) implications category, a genomic findings with companion diagnostic (CDx) category, a targeted therapies with pan-tumor indications category, or an investigational targeted therapies category.

5. The method of claim 1, wherein the medical information includes relative clinical significances based on genomic information, and wherein the patient-specific medical data is ranked according to the relative clinical significances based on the patient genomic testing data.

6. The method of claim 1, further comprising:
comparing the patient genomic testing data to data having diagnostic implications;
determining whether the patient genomic testing data satisfies a first predetermined criteria based on the data having diagnostic implications, wherein the first predetermined criteria comprises a match of one or more of a gene variant or a biomarker in the medical information;
based on a determination that the patient genomic testing data and patient tumor type satisfies the first predetermined criteria, generating the patient-specific data to include genomic findings with the diagnostic implications, wherein medical information includes an annotation indicative of a gene variant and a tumor type, wherein the first predetermined criteria comprises a match of the one or more of the gene variant or the biomarker in the medical information to the annotation.

7. (canceled)

8. The method of claim 1, further comprising:
comparing the patient genomic testing data and a patient tumor type to medical information in a knowledgebase, wherein the medical information in a knowledgebase includes one or more targeted therapies; and
determining whether the patient genomic testing data and the patient tumor type satisfies a second predetermined criteria based on the medical information in the knowledgebase, wherein the second predetermined criteria comprises a match of the patient tumor type and a gene variant or biomarker in the patient genomic testing data with medical information in the knowledgebase for approved targeted therapies with particular clinical significance;
based on a determination that the patient genomic testing data and the patient tumor type satisfies the second predetermined criteria, generating patient-specific medical data of clinically significant targeted therapies with potential clinical benefit;
if the patient genomic testing data and the patient tumor type satisfies the second predetermined criteria for more than one of the clinically significant targeted therapies, reducing the patient-specific medical data to include only approved therapies with the highest level of evidence, wherein the medical information in the knowledgebase for approved targeted therapies with particular clinical significance includes an annotation, and wherein the second predetermined criteria comprises a match of the patient tumor type and the gene variant or biomarker to the annotation.

9. (canceled)

10. The method of claim 1, further comprising:
comparing the patient genomic testing data and a patient tumor type to medical information in a knowledgebase for targeted therapies with expected resistance;
determining whether the patient genomic testing data and the patient tumor type satisfies a third predetermined criteria based on the medical information in the knowledgebase, wherein the third predetermined criteria comprises a match of the patient tumor type and a gene variant or biomarker in the patient genomic testing data with medical information in the knowledgebase for targeted therapies with expected resistance with particular clinical significance;
based on a determination that the patient genomic testing data and the patient tumor type satisfies the third predetermined criteria, generating patient-specific medical data of clinically significant targeted therapies with expected resistance, wherein the medical information in the knowledgebase for targeted therapies with expected resistance with particular clinical significance include an annotation, and wherein the third predetermined criteria comprises a match of the patient tumor type and the gene variant or biomarker to the annotation.

11. (canceled)

12. The method of claim 1, further comprising:
comparing the patient genomic testing data and a patient tumor type to data in a knowledgebase for genomic findings with non-targeted therapy implications;
determining whether the patient genomic testing data and the patient tumor type satisfies a fourth predetermined criteria based on the data in the knowledgebase, wherein the fourth predetermined criteria comprises a match of the patient tumor type and a gene variant or biomarker in the patient genomic testing data with data in the knowledgebase for genomic findings with non-targeted therapy implications with particular clinical significance;
based on a determination that the patient genomic testing data and the patient tumor type satisfies the fourth predetermined criteria, generating patient-specific medical data of clinically significant genomic findings with non-targeted therapy implications.

13. The method of claim 12, wherein the data in the knowledgebase for genomic findings with non-targeted therapy implications with particular clinical significance, and wherein the fourth predetermined criteria comprises a match of the patient tumor type and the gene variant or biomarker to the annotation.

14. The method of claim 1, further comprising:
comparing the patient genomic testing data, a patient tumor type, and a patient age to clinical trial data in a knowledgebase;
determining whether the patient genomic testing data, the patient tumor type, and the patient age satisfies a fifth predetermined criteria based on the clinical trial data in the knowledgebase, wherein the fifth predetermined criteria comprises a match of the patient tumor type, the patient age, and a gene variant or biomarker in the patient genomic testing data with the clinical trial data in the knowledgebase;
based on a determination that the patient genomic testing data, the patient tumor type, and the patient age satisfies the fifth predetermined criteria, generating patient-specific medical data of clinically significant evidence-matched clinical trials.

15. The method of claim 14, wherein the fifth predetermined criteria is indicative of a gene variant or biomarker, a tumor type, a patient age, and a clinical trial patient recruitment criteria.

16. The method of claim 1, further comprising:
comparing the patient genomic testing data and a patient tumor type to data in a knowledgebase for genomic findings with prognostic implications;
determining whether the patient genomic testing data and the patient tumor type satisfies a sixth predetermined criteria based on the data in the knowledgebase, wherein the sixth predetermined criteria comprises a match of the patient tumor type and a gene variant or biomarker in the patient genomic testing data with data in the knowledgebase for genomic finding with prognostic implications with particular clinical significance;
based on a determination that the patient genomic testing data and the patient tumor type satisfies the sixth predetermined criteria, generating patient-specific medical data of clinically significant genomic findings with prognostic implications, wherein the data in the knowledgebase for genomic finding with prognostic implications with particular clinical significance include an annotation, and wherein the sixth predetermined criteria comprises a match of the patient tumor type and the gene variant or biomarker to the annotation.

17. (canceled)

18. The method of claim 1, further comprising:
comparing the patient genomic testing data and a patient tumor type to data in a knowledgebase for genomic findings with potential germline implications;
determining whether the patient genomic testing data and the patient tumor type satisfies a seventh predetermined criteria based on the data in the knowledgebase, wherein the seventh predetermined criteria comprises a 1) a match of a potential germline mutation associated with one or more hereditary cancer predisposition syndromes associated with germline implications in the patient genomic testing data, and 2) a determination that the potential germline mutation is above a predetermined variant allele frequency (VAF) threshold in the patient genomic testing data;
based on a determination that the patient genomic testing data and the patient tumor type satisfies the seventh predetermined criteria, generating patient-specific medical data of clinically significant genomic findings with potential germline implications to be considered for additional germline testing in a clinical context, wherein the data in the knowledgebase for genomic findings with potential germline implications include an annotation, and wherein the seventh predetermined criteria comprises a match of at least the potential germline mutation associated with one or more hereditary cancer predisposition syndromes associated with germline implications to the annotation.

19. (canceled)

20. The method of claim 1, wherein generating the report comprises generating a companion diagnostic (CDx) report associated with the patient.

21. The method of claim 1, further comprising causing an electronic device to display the report, wherein the electronic device comprises a human machine interface (HMI) associated with a clinician or an electronic device associated with the patient.

22. (canceled)

23. (canceled)

24. The method of claim 21, further comprising causing the electronic device to display the report including a predefined area for one or more natural-language phrases on a first page of the report, and wherein the one or more natural-language phrases comprises an embedded hyperlink associated with the one or more natural-language phrases.

25. (canceled)

26. (canceled)

27. (canceled)

28. The method of claim 1, further comprising causing, by the one or more processors, an electronic device to display the report including one or more natural-language phrases in a second predefined area, and wherein the second predefined area comprises an appendix on one or more rearward pages of the report.

29. (canceled)

30. (canceled)

31. A system for generating a report of genomic and medical information associated with a patient, comprising:
one or more processors;
one or more memories; and
one or more programs, wherein the one or more programs are stored in the one or more memories and configured to be executed by the one or more processors, the one or more programs including instructions for:
receiving, at one or more processors, genomic testing data associated with the patient;
based on the genomic testing data, retrieving, at the one or more processors, medical information including one or more potential clinical treatments for the patient;
determining, by the one or more processors, that the medical information has at least some clinical significance to the patient;
based on at least a portion of the medical information having at least some clinical significance to the patient, generating, by the one or more processors, patient-specific medical data;
determining, by the one or more processors, at least one specific position to dispose the medical information on the report based on the patient-specific medical data; and
generating, by the one or more processors, the report based on the determined at least one specific position.

32. A non-transitory computer-readable storage medium storing one or more programs for generating a report of genomic and medical information associated with a patient, the one or more programs comprising instructions, which when executed by one or more processors of an electronic device, cause the electronic device to:
receiving, at one or more processors, genomic testing data associated with the patient;
based on the genomic testing data, retrieving, at the one or more processors, medical information including one or more potential clinical treatments for the patient;
determining, by the one or more processors, that the medical information has at least some clinical significance to the patient;
based on at least a portion of the medical information having at least some clinical significance to the patient, generating, by the one or more processors, patient-specific medical data;
determining, by the one or more processors, at least one specific position to dispose the medical information on the report based on the patient-specific medical data; and
generating, by the one or more processors, the report based on the determined at least one specific position.
